(19) United States
(12) Patent Application Publication
(10) Pub. No.: US 2019/0083782 A1
(43) Pub. Date: Mar. 21, 2019

(54) IONTOPHORETIC SYSTEMS, KITS AND METHODS FOR TRANSDERMAL DELIVERY OF COSMETIC AGENTS

(71) Applicant: InCube Labs, LLC, San Jose, CA (US)

(72) Inventor: Mir A. Imran, San Jose, CA (US)

(21) Appl. No.: 16/134,445

(22) Filed: Sep. 18, 2018

Related U.S. Application Data
(60) Provisional application No. 62/560,178, filed on Sep. 18, 2017.

(51) Int. Cl. A61N 1/32; A61N 1/30; A61N 1/04

(52) U.S. Cl. CPC A61N 1/325; A61K 9/0009; A61N 1/0448; A61N 1/30

(57) ABSTRACT

Embodiments provide systems for delivering cosmetic agents (CA) into the skin. The system includes a power source and at least two electrode assemblies (EA) which may be attached or otherwise incorporated into a facemask assembly that fits over the user's face. Each EA is configured to be held in contact with a facial or other skin layer. Additionally, each EA includes an electrode that is coupled to the power source to receive an output current from the power source. At least one of the EAs in the pair includes a medium that carries a CA, the medium being provided on the at least one electrode assembly to enable the output current to deliver the cosmetic agent into the epidermal dermal or other layer of the skin. Embodiments are particularly useful for delivering CAs into a selected layer of the skin to produce a desired cosmetic effect.

### CROSS-REFERENCE TO RELATED APPLICATIONS

This application claims the benefit of priority of U.S. Provisional Patent Application Ser. No. 62/560,178 (Attorney Docket No. ICUB.P053P), entitled “Iontophoretic Device And System For Transdermal Delivery Of Cosmetic Agents”, filed Sep. 18, 2017, which is fully incorporated herein by references for all purposes.

This application is also related to concurrently filed co-pending U.S. Patent Application Ser. No. ______ (Attorney Docket No. ICUB.P053-1), entitled “Iontophoretic Systems And Methods For Transdermal Delivery Of Cosmetic Agents”, filed Sep. 17, 2018 which is fully incorporated by reference herein for all purposes.

This application is also related to U.S. Pat. No. 8,190,252, entitled “Iontophoretic System For Transdermal Delivery Of Active Agents For Therapeutic And Medicinal Purposes”, filed Aug. 6, 2009; which is fully incorporated by reference herein for all purposes.

### FIELD OF THE INVENTION

Embodiments described herein relate to iontophoretic transdermal delivery of active agents. More specifically, embodiments of the invention relate to the iontophoretic transdermal delivery of active agents such as cosmetic agents. Still more specifically, embodiments of the invention relate to iontophoretic transdermal delivery of cosmetic agents using a conformal patch which fits over portions of the face.

### BACKGROUND

Iontophoresis is a non-invasive method of propelling high concentrations of a charged substance, known as the active agent, transdermally by repulsive electromotive force using a small electrical charge. This method has been used for the transdermal delivery of various compounds including therapeutic agents. Traditionally, direct current has been used to provide the driving current for iontophoresis. However there are a number of short comings associated with the use of direct current including limitations on the total amount of current that can be delivered over time without causing injury to the skin, as well as the buildup of capacitive charge in the skin layer which can oppose the electromotive driving forces thus reducing the rate and total amount of compound delivered over time. Also direct current can cause a local anesthetic effect to the skin resulting in burns and other thermal damage to the skin because the user doesn't feel the injury to the skin occurring at the time. Thus, there is need for improved methods for delivering various therapeutic agents using transdermal iontophoresis.

Many cosmetic agents such as moisturizers, etc. suffer the drawback from not being able to penetrate the surface or epidermal layer of the skin, where they would be more effective. Thus, there is a need for a methods for the transdermal delivery of various cosmetic agents. Iontophoretic transdermal delivery may be one solution however, as described above the method could result in irritation, burns or other injury to the very area of the skin sought to be treated by the cosmetic agent. Thus, there is a need for delivering cosmetic agents to the skin using transdermal iontophoretic agents in a manner which would not cause irritation, burns or other injury to the skin.

### BRIEF DESCRIPTION OF THE INVENTION

Various embodiments of the invention provide an iontophoretic system for transdermal delivery of active agents. Iontophoresis is a non-invasive method of propelling high concentrations of a charged substance, known as the active agent, transdermally using electrical current applied at the skin layer. The active agent can include a drug or other therapeutic agent or biological compound. In many embodiments, the active agent comprises one or more cosmetic agents. In these and related embodiments, the cosmetic agents may be contained and transdermaly delivered to the user's skin by means of a face mask described herein so as to produce a cosmetic effect in the skin of the user contacted by the mask as well as the surrounding skin.

In a first aspect, embodiments of the invention provide a system for transdermal delivery of active agents including cosmetic agents for the treatment of the patient's skin at one or more locations on the patient's body including, for example, the face, head and neck. The system includes a power source and at least one electrode assembly. The power source provides an output current to the at least one electrode assembly that alternates between a maximum current value and a minimum current value. In many embodiments, the system includes at least two electrode assemblies. In specific embodiment the at least two electrode assemblies are configured as a pair of electrode assemblies. Each electrode assembly is configured to be held in contact with a skin layer of a user such as that on the user's face. Additionally, each electrode assembly includes an electrode that is coupled to the power source to receive the output current from the power source. At least one of the electrode assemblies in the pair includes a medium that carries a cosmetic or other active agent having a charge, the medium being provided on the at least one electrode assembly to enable the output current to repel the active agent into a selected skin layer (e.g., the dermis) for a duration in which the output current has a polarity that is the same as a polarity of the active agent. According to one or more embodiments, the output current is a charged balanced alternating current (AC) output.

In many embodiments, the electrode assemblies are positioned on the inner surface of a mask shaped and configured to fit over all or a portion of a user's face (herein a face mask). Collectively, the facemask and the electrodes assemblies will now sometimes be referred to as a facemask assembly or facial treatment mask. Desirably, the face mask assembly is configured to conform to the contour of the user's face as well as bend and flex with movement of the user's face such the electrode assemblies maintain electrical contact with the user's skin including during periods of current delivery. In one or more embodiments, the electrode assemblies can be arranged to fit over the left and right half of the user's face and may be separated by an insulative barrier such as silicone. The electrode assemblies will typically comprise a hydrogel layer which contacts tissue, an electrode and an insulative layer above the electrode. The electrode will typically comprise graphite or other conductive material that is sandwiched between the hydrogel and the insulative layer. Desirably, the electrode assembly including the electrode are sufficiently flexible to allow the facemask assembly to bend and flex with movement of the user's face so as to allow the electrode assemblies to maintain electric contact with the users skin. The insulative material is located above the electrode any may comprise any insulative material known in the polymer and medical arts. It may also include indicia or other markings on its top surface (that facing away from tissue) indicating what the cosmetic agents(s) is/are in the facemask as well as the dosage. For embodiments including the facemask assembly, the power source may be incorporated into or otherwise coupled to the facemask assembly along with control electronics such as a waveform shaper, timer, interface etc. (one or more of which may incorporated into an electronic controller) described herein along with buttons, rocker switches or other input mechanism or means for the user to select a delivery regimen and/or initiate delivery of current from the power source to the electrode assemblies and delivery of the cosmetic agent into the skin. Alternatively, the power source may be external to the facemask assembly, with configured to be connectable to external power source using connection means known in the art.

Desirably though not necessarily, the hydrogel layers is sticky on both sides so as to stick to both the skin as well as the electrode which can be implemented through the use of various adhesives known in the medical/adhesive arts. It is impregnated or otherwise contains the cosmetic or other active agent. It will also typically contain a liquid carrier medium such as an aqueous solution in which the cosmetic agent is or becomes dissolved. In these and related embodiments the hydrogel layer may include a pH buffering agent to maintain a neutral pH of the carrier medium. In some embodiments, the electrode assembly including the hydrogel layer may be fluidically coupled to a reservoir of the medium, buffering agents and one or more electrolytes to increase the conductivity of the solution. As an additional feature in such embodiments, the assembly can include a releasable tab or seal which the user pulls before use to allow fluid to flow from the reservoir into the hydrogel layer. Once there, the hydrogel wets and swells with the solution. In one implementation, the cosmetic agent is still contained in the hydrogel layer and then dissolves in the carrier medium once the medium reaches the hydrogel. In another implementation, the cosmetic agent along with the buffering agents are pre-dissolved in the carrier medium in the reservoir and the mixed solution flows into and wet the hydrogel.

In various embodiments, the active agent contained in the electrode assembly/facemask assembly may correspond to various moisturizing agents, anti-oxidants, anti-wrinkle agents, collagen stimulating agents, skin lightening agents or acne treatment agents. In specific embodiments the cosmetic agent may comprise one or more peptides having inhibitor effects on motor neuron which innervate the facial region so to reduce the appearance of lines and wrinkles by relaxing muscles in the face which causes the wrinkles. According to one or more embodiments, the cosmetic agent(s) may be preloaded into the electrode assembly and/or reservoir. In such embodiments, the user selects the facemask containing the desired cosmetic agent. In alternative or additional embodiments, the user may load the cosmetic agent or cosmetic agent solution into the electrode and/or facemask assembly. In such embodiments, cosmetic agent solution can be contained in bottle or other container which has dispensing tip or other dispensing element configured to be fluidically coupled to one or more of the electrode assembly, hydrogel or reservoir.

In a second aspect, the invention provides a kit comprising an embodiment of the facemask assembly described herein preloaded with one or more cosmetic agents described herein which is packaged in sealed packaging. In some embodiments, the kit may also include a bottle or other container of cosmetic agent solution, such as that described above, which the user loads into the facemask or electrode assembly. The kit may include a single facemask assembly comprising one cosmetic agent or multiple facemask assemblies which comprise the same or different cosmetic agents. For multiple face mask assemblies, the facemasks may be configured to provide a facial treatment regimen. In such embodiments the treatment regimen comprises the serial application of facemasks and delivery of cosmetic agent over a selected period of time. The facial treatment regimen may be implemented through the selection of the particular cosmetic agent and/or the dose of cosmetic agent.

In a third aspect, the invention provides methods of iontophoretically delivering a cosmetic or other active agent to the skin of the user's face or other location on their body using an embodiment of the facemask assembly. According to one embodiment of such a method a facial treatment mask is made provided to the user, the mask configured to conform to a contour of the user's face and including at least a first and second electrode assembly, each of the electrode assemblies carrying a cosmetic agent having a charge. The mask is then applied to the user's face wherein the mask conforms to the contour of the user's face such that the first and second electrode assemblies uniformly contact the user's facial skin. Alternating current is then generated and delivered through each of the first and second electrode assemblies to alternatively repel the cosmetic agent from the respective first and second electrode assemblies into the user's facial skin.

The cosmetic agent can be selected so as to produce a desired cosmetic effect in the facial skin of the user such as skin rejuvenation which may include one or more of wrinkle reduction; lightening of the skin and/or reduction of areas of pigmentation or hyperpigmentation (e.g. agent spots); increased skin thickness; increased skin collagen content, increased skin elasticity or increased skin moisture content. The user may choose the facemask assembly with one or more cosmetic agents or they may add the desired cosmetic agents using a bottle or other application means. The power source for the electrode assemblies may be integrated into/with the mask assembly or may be connectible; if the later, they then connect the facemask assembly to the power source. They then select a delivery period and/or regimen using a button or other input mechanism or means on the facemask assembly or alternatively, using a remote input means such as a cell phone which is operatively coupled to a controller which is part of the facemask assembly. Alternatively, the time period can be preprogrammed into the controller. They then apply the facemask to the desired portion of their face. The user may then press a button on the mask to start the transdermal iontophoretic delivery of the cosmetic agent or alternatively, the facemask may itself initiate delivery of the cosmetic agent by detection by sensors coupled to the mask which detect a change in impedance or other property after the mask has been applied to the user's face. Typically, the cosmetic agent will be delivered by both electrode assemblies using double point dispersion approach described herein. Though in some embodiments, the cosmetic agent is delivered using only one electrode assembly using a single point dispersion approach. In particular embodiments, delivery of cosmetic agents by either one or both electrode assemblies may be selectable by the user. Also, in particular implementations of double point dispersion the amount of cosmetic agent at delivered into the skin at each electrode assembly is configured to be substantially equivalent. In one or more implementations this can be accomplished by setting the on off times for each electrode assembly to be substantially the same.

According to one or more embodiments, the user may treat the skin with a moisturizing agent prior to application of the facemask and subsequent delivery of current. The pretreatment with the moisturizer herein referred to as “pre-moisturization” serves to reduce the impedance of the skin and in turn, reduces the amount of resistive heating of the skin. In use, such embodiments reduce the risk of thermal irritation and/or injury to the skin as well as any resulting erythema or other discoloration of the skin. The moisturizing agent may be supplied with a kit including the facemask assembly or the user may use their own moisturizing agent. In some embodiments, the user may measure the impedance and/or level of hydration of the target skin site to be treated and then, use that information to make a determination as whether to apply the moisturizing agent and for how long. The skin impedance level can be measured using the electrode assemblies included with the face mask or by a separate skin impedance/hydration measurement system or sensor. In use, embodiments of the invention which utilize such skin impedance measurement provide the user with the ability to know when and for how long to apply a moisturizing agent so as to reduce the risk of one more of skin irritation, injury, discoloration etc. In other embodiments, the user may treat the skin prior to the application of the facemask and current delivery by exfoliating the skin using an exfoliating agent known in the art to remove the dead layer of cells in the uppermost layer of skin, the stratum corneum, so as to increase the permeability of the skin to the cosmetic agent as well reduce skin impedance, both serving to increase the transport of cosmetic agent into the skin. The exfoliating agent may be supplied with the kit along with a specific cloth to use for doing the exfoliation which may have the exfoliating agent pre-applied to it. Similar to the pre-moisturization step, prior to or after exfoliation, the user may make measurements of skin impedance using the face mask assembly to determine when a sufficient level of exfoliation has been obtained. In use, pre exfoliation serves to increase the delivery of cosmetic agent into the skin as well to do so more uniformly in the area of skin contacted by the face mask assembly.

The above and other features and advantages of embodiments of the invention are described in more detail below with reference to the accompanying drawings.

### BRIEF DESCRIPTION OF THE DRAWINGS

FIG. 1 illustrates an iontophoretic system for transdermal delivery of an active agent, according to one or more embodiments.

FIG. 2 illustrates an alternative embodiment in which each of a pair of electrode assemblies are equipped to disperse an active agent into a skin layer of the user.

FIG. 3 is a top view of the electrode assemblies deployed on a skin layer of the user.

FIG. 4 illustrates an alternating power source for use with embodiments such as described with FIG. 1 though FIG. 3.

FIG. 5A through FIG. 5F illustrate various waveforms or current output variations that can be used to promote a characteristic of the electrode assemblies operation on a user's skin.

FIG. 6A1 illustrates an embodiment of a facemask assembly having electrode assemblies for delivery of cosmetic agents to the facial skin of a patient.

FIG. 6A2 illustrates an embodiment of a facemask assembly having electrode assemblies for delivery of cosmetic agents to the facial skin of a patient where the power source is integral or otherwise directly coupled to the mask.

FIG. 6B is a side view illustrating an embodiment of the facemask assembly.

FIG. 6C is a side view illustrating an embodiment of the facemask assembly fitting onto the profile of a user's face.

FIG. 6D illustrates an embodiment of the facemask assembly having a strap for securing the mask to the user's face.

FIG. 7 is a cross sectional view to illustrate an electrode assembly for delivery cosmetic agents to a skin layer of a patient, according to an embodiment.

FIG. 8A and 8B are side views of a mold for creating a facemask, according to an embodiment.

FIG. 9A, 9B and 9C illustrate the use of photographic images to custom fabricate a face mask, according to one or more embodiments.

FIG. 10 illustrates a facemask assembly kit for delivery of cosmetic agents to a skin of a patient, according to an embodiment.

FIG. 11 is a cross sectional view of a skin layer, illustrating delivery of cosmetic agent to skin layers in increasing and decreasing vertical concentration gradients by application of one or more embodiments, as described herein.

### DETAILED DESCRIPTION OF THE INVENTION

Embodiments described herein provide for an iontophoretic system for transdermal delivery of active agents. As used herein, the term transdermal refers to the delivery of a compound, such as a drug or other biological agent, into and/or through one or more layers of the skin (e.g., epidermis, dermis, etc.). Iontophoresis is a non-invasive method of propelling high concentrations of a charged substance, known as the active agent, transdermally using electrical current applied at the skin layer. All chemical compounds are considered to either have a net charge or a residual charge due to Van der Walls, dipole interactions and other forces. For those compounds that do not have a net charge they can be formulated so they are using known methods in the art such as adding a charged functional group which is described in more detail herein. The active agent can include a drug or other therapeutic agent or biological compound. In many embodiments, the active agent comprises a cosmetic agent. As used herein a “cosmetic agent” is any agent used to treat or improve the health and/or appearance of the skin and may include, for example, various moisturizing agents, anti-oxidants, collagen stimulating agents, anti-wrinkle agents including neurotoxins such as botulinum toxin, sun block agents, or acne treatment agents and skin lightening agents. Also, as used herein the term “about” generally refers to within ±10% of the stated value of a property, dimension, characteristic or other value and more preferably within 5%. Also, as used herein the term “substantially” means within ±10% of a stated property or quality and more preferably, within ±5% of the stated property or quality.

More specifically, embodiments described herein include a system for transdermal delivery of active agents including cosmetic agents for the treatment of one or more locations on the patient's body including the face, head and neck. The system includes a power source and at least one electrode assembles. The power source provides an output current that alternates between a maximum current value and a minimum current value; a pair of electrode assemblies. In many embodiments the system includes at least two electrode assemblies. Each electrode assembly is configured to be held in contact with a skin layer of a user such as that on the user's face. Additionally, each electrode assembly includes an electrode that is coupled to the power source to receive the output current from the power source. At least one of the electrode assemblies in the pair includes a medium that carries a cosmetic or other active agent having a charge, the medium being provided on the at least one electrode assembly to enable the output current to repel the active agent into a skin layer (e.g., the dermis, or epidermis) for a duration in which the output current has a polarity that is the same as a polarity of the active agent.

According to one or more embodiments, an output current such as described is a charged balanced alternating current (AC) output. The charged balance AC output means over a given duration, the amount of current delivered at each polarity is substantially equivalent. As used herein, “substantially equivalent” means that two values are within 80% of one another, more preferably within 90% and still more preferably within 99% over the period of one or more waveforms. This same definition holds for the term “substantially the same”.

Single Point Disbursement

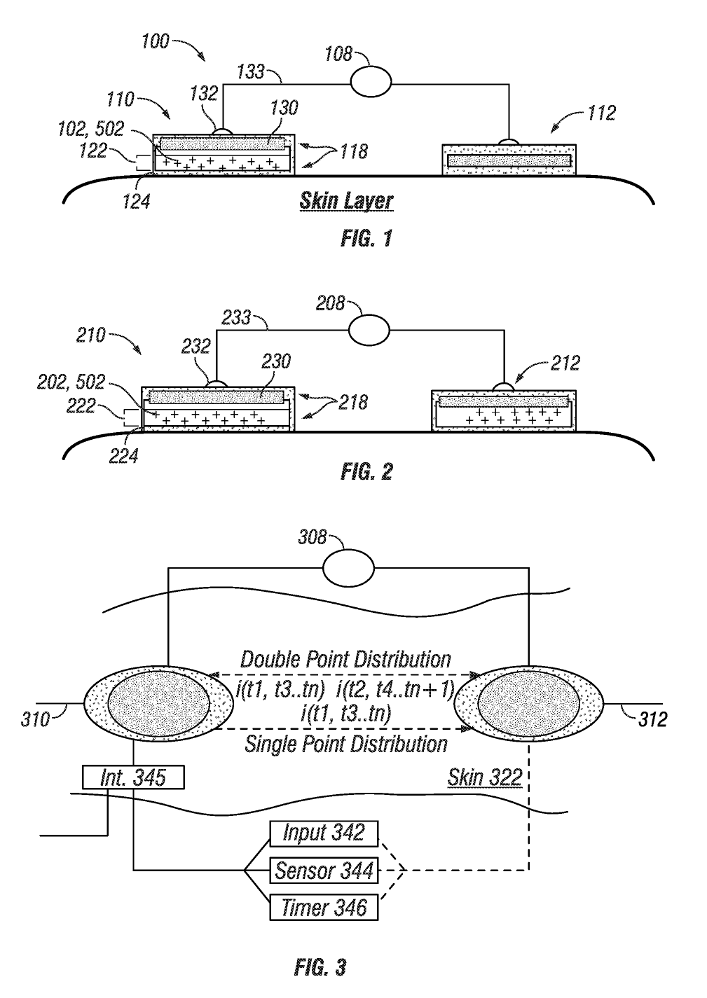

FIG. 1 illustrates an iontophoretic system for transdermal delivery of an active agent such as a cosmetic agent, according to one or more embodiments of the invention. A system 100 is shown in a deployed (i.e., operational) state, and comprises a pair of active electrode assemblies 110, 112 and alternating power source 108 that combine to enable the transdermal delivery of a medicinal or therapeutic (“active”) agent 102 into a user's tissue such as skin tissue. Therapeutic agent 102 can comprise one or more drugs or other therapeutic agents including cosmetic agents. In the deployed state, the pair of electrode assemblies 110, 112 are positioned on the exterior skin layer of the user. In one embodiment, the alternating power source 108 forces the agent 102 to be dispensed from one of the electrode assemblies in the pair (shown as electrode assembly 110 in FIG. 1). More specifically, the active agent 102 is selected to have an ionic charge, and the alternating power source 108 is connected to electrode assembly 110 to repel the active agent 102 into a skin layer of the user at instances when the alternating power source has the same polarity as the active agent. As such, the driving mechanism that causes the active agent 102 to dispense into the skin layer is intermittent and alternating (to match the output of the power source 108).

With specific reference to FIG. 1, the power source 108, electrode assemblies 110, 112 and user skin layer, e.g., a facial skin layer, or other tissue form a circuit to enable delivery of the active agent from at least one of the electrode assemblies. More specifically, FIG. 1 illustrates a single disbursement configuration in which the first electrode assembly 110 contains the active agent, and the second electrode assembly 112 serves as a return without the active agent. In the configuration shown, the second electrode assembly 112 serves as the return for completing the circuit with power source 108 and the first electrode assembly 110. For a duration, the output current is provided a polarity that matches that of the charge of the active agent. The presence of the output current, flowing via the circuit formed by the other electrode assembly and the power source 108, results in the charged active agent being repulsed from the electrode assembly 110 into a skin layer of the user (e.g., the epidermis, dermis or hypodermis). Thus, in a configuration shown by FIG. 1, the first active electrode assembly 110 is equipped with the active agent 102, and the power source 108 directs the active agent from the first electrode assembly 110 into the skin layer when the polarity of the output current matches that of the charge of the active agent.

As described below, the power source 108 may vary the output of the current output to alternate durations in which the active agent is delivered. In one embodiment, the power source 108 varies the output current between a maximum current value (coinciding with a delivery duration) and a minimum current value (coinciding with non-delivery duration). The minimum current value corresponds to either no current output, or a reverse current output. As described elsewhere, the reverse current output may serve as a retention mechanism that actively precludes the active agent from diffusing into the skin layer (e.g., due to electrostatic attractive forces). Thus, a delivery duration coincides with a duration in which an output current from the power source 108 has polarity to match that of the active agent. A non-delivery duration coincides with either an output current from the power source that is opposite in polarity to that of the active agent, or to a duration that coincides with substantially no current output.

In a system 100 such as described with FIG. 1, some embodiments provide for the delivery/non-delivery durations to be symmetrical or equal. For example, delivery/non-delivery durations may each last x milliseconds, seconds, or minutes, to match, for example, symmetrical waveforms of the output (e.g., sinusoidal, square wave etc.). In other embodiments, the delivery/non-delivery durations are asymmetrical or unequal. For example, the delivery duration may last several minutes, and the non-delivery duration may last only seconds or otherwise be less than the delivery duration. The delivery/non-delivery durations may repeat, or pass through only a single cycle (i.e., one delivery duration and one non-delivery duration).

Each electrode assembly 110, 112 includes an electrode 130 and a contact thickness 118. The contact thickness 118 of each electrode assembly 110, 120 may be in the form of a patch fabricated from layers of elastomeric or other flexible polymer material. The contact thickness 118 may include, for example, adhesives for enabling the respective electrode assemblies 110, 112 to be deployed on the skin layer of the user and to remain adhered over an extended period of time during movement of the skin. Likewise, the electrode 130 corresponds to one or more elements or layers that extend the conductive path from the alternating power source to the contact thickness and/or skin layer. In one embodiment, a connector 132 connects the electrode 130 to leads 133 of powers source 108. The electrode 130 corresponds to a metal layer or element(s) (e.g., wiring, contact elements etc.) that extends or connects to the connector 132. The electrode 130 may comprise a separate layer from the contact thickness 118, which includes a medium 122 for carrying the active agent 102. However, in some variations, the electrode 130 includes elements, such as particles or contact elements that are integrated or provided with the contact thickness 118. In one implementation, the electrode 130 is comprised of conductive material, such as metal (e.g., silver) or conductive carbon material (graphite sheets). In an embodiment depicted by FIG. 1, electrode 130 is a conductive layer that overlay the contact thickness 118. As described below, the contact thickness 118 includes thicknesses for dispersing the active agent 102, as well as material to enable the electrode assembly to be adhered to skin. In many embodiments, the active agent is dissolved in an aqueous or other carrier solution, for example, isopropyl alcohol, DMSO and like compounds. For embodiments where the active agent is a cosmetic agent, the cosmetic agent is preferably dissolved in an aqueous solution and may include various buffering agents to allow the solution to be maintained at an overall neutral pH.

As previously mentioned, in an embodiment of FIG. 1, only one of the electrode assemblies in the pair (shown as electrode assembly 110) is used to deliver the cosmetic or other active agent 102 into the user's skin. The medium 122 of the first electrode assembly 110 provides a reservoir or retainer that contains the cosmetic or active agent, for example, in embodiments where the active agent is dissolved in a carrier solution. More specifically, the medium 122 of the contact thickness 118 includes a tissue contacting porous layer 124, which can either be separate or part of a reservoir. The porous layer 124 can be configured to absorb the carrier solution from the reservoir and in turn, wick the solution into contact with the skin (e.g., by capillary action). The porosity of the porous layer 124 may be selected based on various parameters. For example, the porosity may be selected based on the concentration or transport characteristics of the cosmetic or other active agent. More specifically, for example, high porosities can be selected for higher molecular weight cosmetic or other therapeutic agents and/or therapeutic agents solutions having greater viscosity and vice versa. Suitable porous materials for porous layer 124 can comprise compressed cotton or other fibrous meshes such as meshes made from various polymer fibers known in the art.

In various embodiments, the electrode assemblies 110, 112 can be constructed to be disposable or reusable. If disposable, the electrode assembly 110 (carrying the active agent) is manufactured or retailed to include the cosmetic or other active agent in the medium 122 (for example a wrinkle reducing agent). If reusable, an embodiment provides that the electrode assembly 110 includes an intake conduit and optional self-sealing port that enables the active agent 102 to be dispersed in the medium 122 for delivery. In one embodiment, the self-sealing port is formed from silicone or other elastomeric material, so as to enable the electrode assembly 110 to be filled with the active agent.

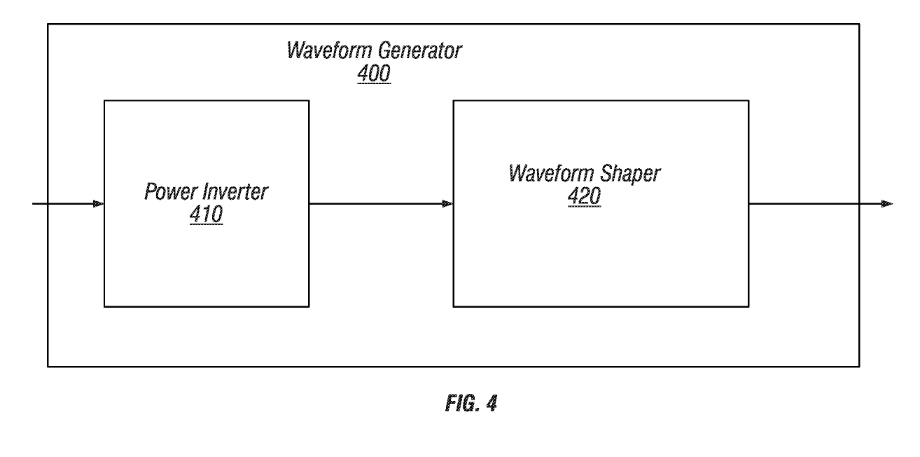

The alternating power source 108 may include a DC power source such as a battery for example a rechargeable Lithium-Ion battery which may be in the form of a battery pack. As an alternative, the alternating power source 108 may, include or provide an interface, to another power source, such as a solar cell. Circuitry (such as described with FIG. 4) may be used to convert the direct-current (DC) power output to an alternating current (AC) signal of a specified waveform. As mentioned elsewhere, the specified waveform may be short (e.g., milliseconds), long (minutes), symmetrical (delivery/non-delivery are equal), or asymmetrical (delivery/non-delivery are now equal).

The electrode assemblies 110, 112 and the alternating power source 108 may be provided in connection with one or more housing segments. For example, the power source 108, electrode assemblies 110, 112, and wiring or connectors that interconnect the power source and the electrode assemblies may all be contained by a housing, or combination of integrated housing segments. In this way, the system of electrode assemblies 110, 112 may be provided as a product, device or kit that can be assembled and deployed by the user. The kit may further include instructions for use. For embodiments using a facemask, the electrodes assemblies are desirably positioned on an inside surface of the facemask so that they are electrically coupled to the skin when the facemask is placed on the face of the user.

When deployed and made operational, the cosmetic or other active agent is selected to have an ionic charge that can be sufficiently repulsed by the presence of current having the same polarity so as to be driven into the subject's skin. The active agent is distributed in the medium 122 of the electrode assembly 110. The power source 108 is connected and signaled, resulting in a circuit being formed between the alternating power source 108, electrode assembly 110 containing the active agent, and the electrode assembly 112 providing the return electrode. In the durations when the current has the same polarity as the charge of the active agent, the active agent is repulsed from the medium 122 of the electrode assembly 110 into a skin layer of the user. In the durations when the current has the opposite polarity as the charge of the active agent, the active agent is not repulsed. Thus, the active agent is induced to travel into the skin layer in alternating durations to match the alternating power of the alternating power source 108. The frequency of the alternating power source 108 may vary greatly. In particular, the frequency of the alternating power source may be in the range of milliseconds (e.g., 1/60 seconds) or minutes (e.g., ten minutes).

Among other benefits to this approach, the diffusion of the cosmetic or other active agent into the skin layer can be completely stopped with the switch in the current polarity. Thus, use of the alternating power source 108 enables the cosmetic or other active agent to be stopped from entering the skin layer at alternating instances. This enables, for example, better control of the amount of cosmetic or other active agent delivered into the skin layer in a given duration. Further stopping of the diffusion can allow the user to observe the effects of the agent to the skin. In use this allows the user to make a decision whether to stop treatment, continue with treatment with the same agent or switch to the use of a different agent,

Double Point Disbursement

FIG. 2 illustrates an alternative embodiment in which each of a pair of electrode assemblies are equipped to disperse an active agent into the skin layer of a user, under another embodiment. More specifically, an embodiment of FIG. 2 shows a first and second electrode assembly 210, 212, each of which can include a construction similar to that shown with the first electrode assembly 110 of FIG. 1. Accordingly, the first and second electrode assemblies 210, 212 each include an electrode 230 positioned over or in operative relationship to a contact thickness 218. The contact thickness 218 of each electrode assembly 210, 220 may be in the form of a patch fabricated from layers of elastomeric or other flexible polymer material. The contact thickness 218 may include, for example, adhesives for enabling the respective electrode assemblies 210, 212 to be deployed on the skin layer of the user. Likewise, the electrode 230 of each electrode assembly 210, 212 may correspond to one or more metal layers or element(s) (e.g., wiring, contact elements, etc.) that extends or connects to a connector 232, which in turn connects that electrode 230 to leads 233 of power source 208. On each electrode assembly 210, 212, the electrode 230 may comprise a separate layer from the contact thickness 218, which includes a medium 222 for carrying the active agent 202. However, in some variations, the electrode 230 includes elements, such as particles or contact elements, that are integrated or provided with the contact thickness 218. In one implementation, the electrode 230 is comprised of conductive material, such as metal (e.g., silver or silver-silver-chloride) or conductive carbon material (e.g., graphite sheets).

The medium 222 of the electrode assemblies 210, 212 includes a tissue contacting porous layer 224, which can either be separate or part of a reservoir. Similarly, in an implementation in which one or both of the electrode assemblies 210, 212 are reusable, a self-sealing port (not shown) may be included to enable the active agent to be dispersed in the medium 222 for delivery to the skin layer.

As a variation, the electrode assemblies 210, 212 may both be capable of retaining the cosmetic or other active agent to dispense, but the electrode assemblies 210, 212 may have differing constructions. For example, the contact layer and amount of cosmetic agent 202 each electrode assembly 210, 212 can retain may be different.

In contrast to an embodiment of FIG. 1, the alternating source 208 is electrically connected to cause dispersion of active agent 202 from both electrode assemblies 210, 212 in alternating fashion. In one embodiment, the alternating power source 208 alternates the power signal to each electrode so that the delivery durations from each electrode assembly are the same. Such a configuration enables delivery durations to alternate between electrode assemblies. Among other benefits, alternating the delivery durations between electrode assemblies enables continuous transdermal delivery of active agents using alternating points in the user's skin, to avoid, for example, skin irritation or saturation. It also allows equal delivery of cosmetic agent to each electrode so as to produce a more uniform cosmetic effect on the skin.

Similar to prior embodiments of FIG. 1, an embodiment such as described with FIG. 2 may be constructed as a device or kit that can be assembled and deployed for use by the user. Accordingly, one or more housing segments may be incorporated to integrate the electrode assemblies 210, 212 and/or power source 208.

FIG. 3 is a top view of the electrode assemblies deployed on a skin layer of the user. The electrode assemblies 310, 312 may be implemented to disperse an active agent from one electrode assembly (single point disbursement, such as described with FIG. 1) or from both electrode assemblies 310, 312 (double point disbursement, such as described with FIG. 2). In a single point disbursement configuration, the alternating power source 308 repulses the active agent into the skin 322 (into the paper, as depicted by Z axis) in alternating durations when the supplied current has the same polarity as the charge of the active agent. As mentioned elsewhere, the alternating durations may last milliseconds, seconds, or minutes. The alternating durations may also be asymmetrical or unequal in duration. In a single point disbursement, for example, current is extended from the alternating power source 308 through the contact thickness (see element 118 of FIG. 1) of the first electrode assembly 310, into the skin layer 322, and to the second electrode 312 (serving as the return) to form a circuit with the alternating power source 308. The active agent is thus dispensed from one electrode assembly 310 into the skin layer in alternating durations (durations marked by t₁, t₃, tₙ) set by the frequency of the current from the power source 108. Significantly, the active agent does not dispense passively in the alternating instances when the polarity of the current is opposite to the charge (i.e., attractive polarity) of the active agent (durations marked by t₂, t₄, tₙ₊₁). In that instance, the opposite polarity of the current/voltage serves as a retention mechanism of the active agent within the electrode assembly 310.

In a double point disbursement configuration (such as described with an embodiment of FIG. 2), the alternating power source 308 alternates which electrode assembly is directing the active agent into the skin layer 322. In one implementation, for example, both electrode assemblies may carry the active agent, and the active agent is positively charged. At a first duration when the current has a positive polarity, (i) a positively charged active agent in the first electrode assembly 310 is directed into the skin layer, (ii) a positively charged active agent in the second electrode assembly 312 is retained, or precluded from being diffused into the skin layer. In the next duration, when the current has the negative polarity, (i) a negatively charged active agent in the first electrode assembly 310 is retained or precluded from being diffused into the skin layer; and (ii) a positively charged active agent in the second electrode assembly 312 is directed into the skin layer. The timing sequence of the first electrode assembly 310 thus may be described as (i) dispense at durations marked by (t₁, t₃, tₙ), and (ii) retain at durations marked by (t₂, t₄, tₙ₊₁). Likewise, the timing sequence of the second electrode assembly 312 may be described as (i) dispense at durations marked (t₂, t₄, tₙ₊₁) and (ii) retain at durations marked by (t₁, t₃, tₙ).

With regard to either the single or double point disbursement configuration, the frequency of the electrode assemblies operation may be measured in milliseconds, seconds or minutes. For example, in a single disbursement embodiment, a drug-on mode (e.g., a cosmetic agent on mode) of operation may last several minutes, followed by a drug-off mode. The time periods for the drug-on and drug-off states may be the same or different. For example, the drug-on states may last several minutes, but the drug-off state may be much shorter.

According to an embodiment, the electrode assemblies 310, 312 can be used in connection with the following inputs to initiate and/or stop use of the electrode assemblies: (i) input from a user input mechanism 342, (ii) input from a sensor 344 or sensor system for detecting a human/physiological condition (e.g., skin impedance), and/or (iii) input from a timer 346. A user input mechanism may correspond to a switch, button or similar mechanism that the user can trigger. The user input mechanism 342 may be used to initiate use of the electrode assemblies 310, 312 once the user places the electrode assemblies on his or her skin. The user input mechanism 342 may also be used to stop the electrode assemblies at the user's election. For example, the user may deploy the electrode assemblies on his or her facial or other skin layer, then press a button 342 to cause the power source to power the electrodes at a desired time and the press the same or a different button to depower the electrodes and stop the delivery of cosmetic agent. In embodiments involving the use of facemask assembly 560, the input mechanism 342 may be integral to or otherwise directly coupled to the facemask 550 and may comprise a button or switch positioned such that the user can see and press when the facemask is on the user's face.

The sensor 344 (or sensor system) may correspond to a physiological sensor that triggers the electrode assemblies to operate when the sensor 344 detects a physiological condition or event. For example, the sensor 344 may correspond to a glucose monitor for diabetics; the glucose conditions trigger sensor 344 to actuate the electrode assemblies. In other embodiments, the trigger sensor 344 may correspond to a sensor whose signal is used to initiate delivery of a cosmetic or other active agent 202 when the electrode assemblies are positioned in contact with user's skin such as when facemask assembly 560 is placed on the user's face. Such sensors may correspond to one or more of a pressure sensor, temperature sensor, impedance sensor, capacitance sensor or the like. In the case of the pressure sensor, the sensor can be configured and positioned so sense physical contact of the facemask to skin by an amount of force applied from the mask to the skin. In the case of an impedance sensor, one or both of electrical and physical contact can be sensed by a change (e.g. a reduction) in impedance and in particular embodiments, electrode assemblies 310 and 320 themselves can be configured as such sensors where the sensed impedance is between the electrode assemblies.

As an alternative or variation, a system such as described with FIG. 3 may be provided with an interface 345 to enable the power source 308 to be triggered to start, stop or modulate output of current to the electrode assemblies responsive to the output of sensor 344 or other sensor. In this way, a system such as described by various embodiments may be deployed in an environment where the user has one or more pre-existing body sensors to detect various conditions including various conditions. For embodiments, employing facemask assembly 550, the sensors may be positioned on skin contacting surface of the facemask other couple to facemask to be directly or otherwise operably coupled to the skin's surface when the mask is placed on the face of the user. The interface 345 may include logic or circuitry to enable interpretation of the sensor output from the user's sensor system. In particular embodiments, interface 345 may correspond to a microprocessor or other electronic controller digital or analogue.

The timer 346 corresponds to a mechanism, implemented by, for example, logic or circuitry, that (i) switches the power source 308 from a state of delivery (i.e., signal current output to the electrode assemblies) to a state of non-delivery through current/voltage output; and/or (ii) switches the power source 308 from a state of non-delivery (i.e., signal reverse current or no current) to a state of delivery. In a typical implementation, the timer 346 may switch the power source 308 into a state in which the current output matches the charge of the active agent for a set duration, then switch the power source to either turn off or output a reverse current.

As an alternative or variation to embodiments described, the sensor 344 or a sensor system (which may corporate multiple sensors 344) is configured to trigger the power source 308 to cease or otherwise modulate the delivery of current to electrode assemblies 310 and 320 when a physiological condition or parameter is or is no longer present. In embodiments of the former case, sensors 344 can be configured to detect increases in skin temperature or skin impedance, or skin redness preceding or occurring due to injury to the skin. In these embodiments, sensors 344 may correspond to one or more of a temperature sensor, infrared sensor, impedance sensor or optical sensor (e.g., a charge couple display, aka a CCD). Embodiments of the latter case may include colorimetric sensors. In still another variation, rather than switch off, an embodiment may switch the mode of operation of the electrode assemblies from a drug deliver to a drug-off state. The drug-off state differs from an off state, in that a reverse current may be used to (i) maintain the electrodes in the deployed state, but (ii) retains the active agent with the electrode as a result of the polarity of the current. For example, with reference to an embodiment of FIG. 1, when the sensor 344 detects presence of the physiological condition, the electrode assembly 310 switches on to deliver a type of active agent to address the condition. After the physiological condition is being detected as being treated (either by sensor or timer), the electrode assembly 310 switches into a reverse current state, so that no drug is delivered into the skin layer. Subsequent re-occurrence of the condition may trigger the first electrode assembly 310 into the drug delivery mode again upon the sensor 344 detecting re-occurrence of the physiological condition.

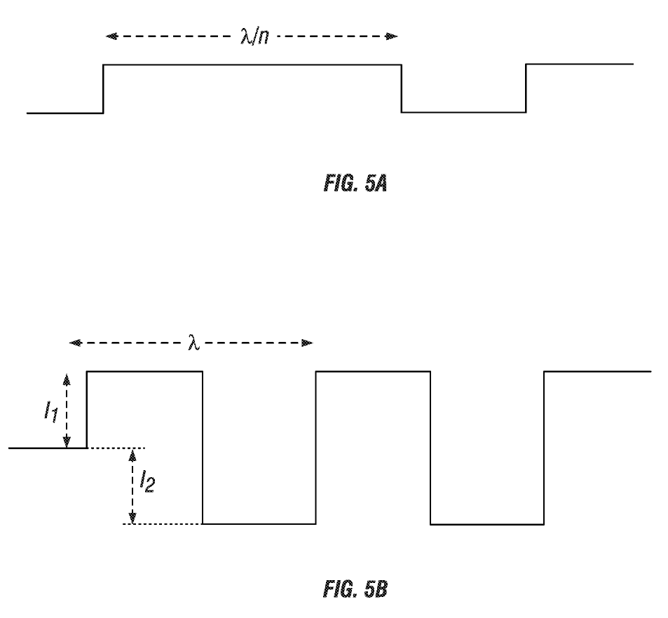

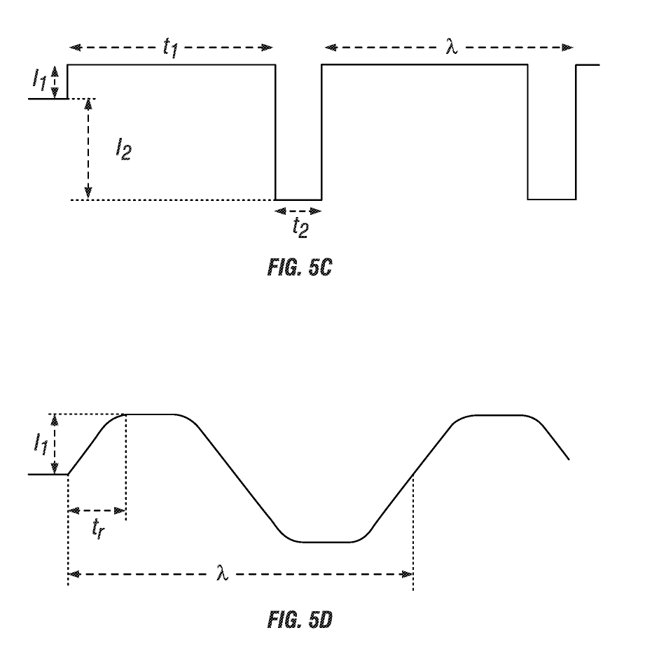

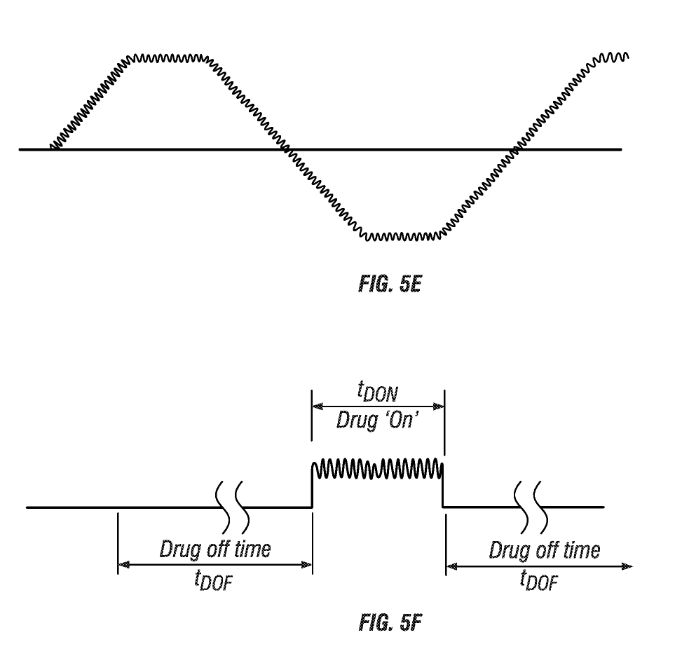

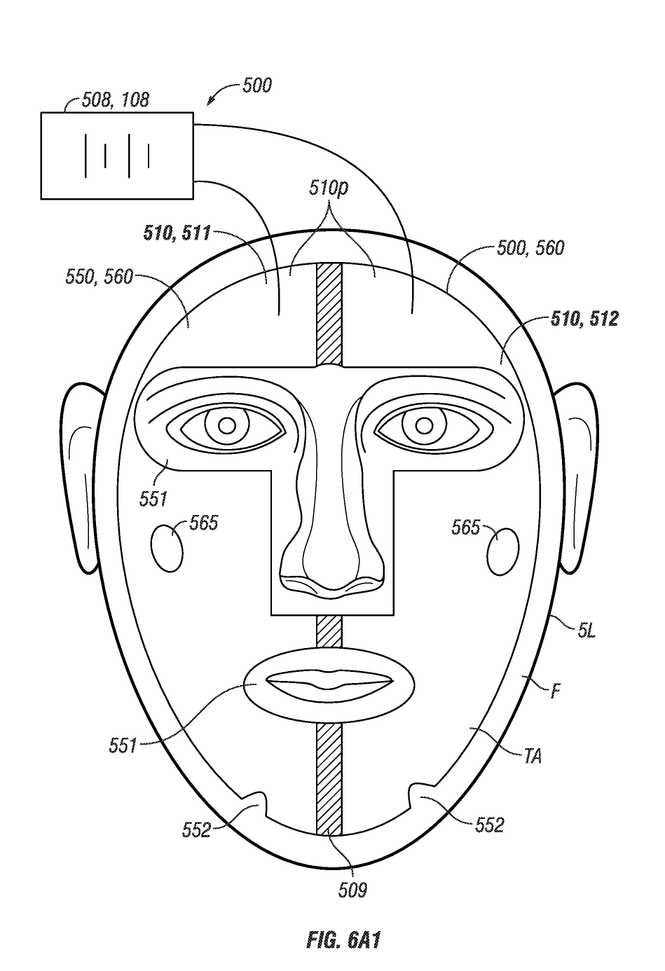

Various embodiments described above provide for alternating current/voltage to drive a charged active agent from an electrode assembly into the skin layer of the user including a facial skin layer. Embodiments further recognize that a waveform of the alternating current/voltage that is output from the alternating power source may be of consequence as to the operation and application for the transdermal iontophoretic delivery system described by various embodiments. Numerous current output waveforms and applications for using such waveforms are described with FIG. 5A through FIG. 5F.

Applications and Waveforms

FIG. 4 illustrates an alternating power source for use with embodiments such as described with FIG. 1 though FIG. 3 so as to generate and deliver an AC or other current to electrode assemblies 310 and 320 or other electrode assemblies such as assemblies 511 and 512 on facemask assembly 560 described herein. In these embodiment the respective power source (e.g., 108, 208, 308 or 508) includes a waveform generator 400 has an input to receive a DC current from a battery (or other power source, such as photovoltaic solar cell) and converts the input into a shaped waveform. Examples of the shaped waveform may include a sinusoidal waveform, a square waveform, a trapezoidal waveform, or other similar waveforms. Some waveforms, such as square waves, in particular, may short or long frequency. Short frequency waveforms may repeat several times per second (e.g., 1/60 seconds), while long frequency waveforms may repeat once over several minutes (e.g., 5, 10 or 20 minutes). In generating the waveforms, some embodiments use a voltage that is in range of 1 to 100 volts.

The waveform generator 400 includes a power inverter 410 and waveform shaper 420. Power inverter 410 has an input to receive the DC current and an output to transmit an AC current to the waveform shaper. The waveform shaper 420 includes circuitry to shape the AC current to the desired waveform. For example, the waveform shaper 420 may include capacitive or inductive elements in order to obtain the desired shape of the waveform. The shaped waveform is then outputted by the waveform generator 400 to electrode assemblies 310 and 320.

FIG. 5A through FIG. 5F illustrates various waveforms or current output variations (over time) that can be used to promote a characteristic of the electrode assemblies operation on a user's skin such as the user's face. Embodiments such as described may be implemented in either a single (see FIG. 1) or double (see FIG. 2) disbursement configuration. In describing an embodiment of FIGS. 5A-5F, reference may be made to elements or numerals of FIG. 3 for purposes of illustration. Numerous embodiments described herein provide for waveforms that vary between a given polarity and zero, wherein at polarity, the current causes the active agent to repel in the skin layer. In other embodiments, the waveforms have alternative between positive and negative polarity. In some embodiments, the alternating currents can be delivered to each electrode assembly that is in use (whether or not the electrode assembly has the active agent). By orienting the waveform to alternate in a charged-balance fashion, electrical toxicity or other damage to the skin can be reduced or minimized. In other embodiments, an alternating current is used that is oriented towards being balanced in charge, but some asymmetry may exist.

The waveforms described below are variable between a minimum and maximum value. Some embodiments, such as described with FIG. 5B, may be alternating in charge value (i.e., include reverse polarity). In such embodiments, the current delivery may be balanced in charge.

FIG. 5A illustrates a waveform 510 that includes an extended or long drug delivery phase, according to an embodiment. In some embodiments, the skin layer may be assumed to handle only a maximum amount of current in a given duration (max current delivery) (e.g. 80 milliamps per minute). For a given amperage, the duration of the output of the alternating power source may be set to not exceed the max current delivery. The delivery duration may be set to some portion or fraction (e.g., 50% for n=2) of the overall period of the current output I₁. For example, in some implementations, the max current delivery (I₁) is assumed to be 80 milliamps for one minute. In such an implementation, the delivery duration is set for 20 seconds on 4 milliamp output. Rather than switch to negative polarity, the output of the power source 308 may alternate to no amperage output (rather than switch polarity). While the waveform depicted in FIG. 5A is rectangular, the waveform may have an alternative shape (e.g., sinusoidal, trapezoidal), with the current delivery corresponding to the area under the curve. In the example shown by FIG. 5A, the alternating power source 308 initiates a delivery duration on one electrode, with delivery durations being set by a current that has a polarity that matches that of the charge of the active agent. The current may alternate to zero output, in which the drug delivery is substantially ceased. Thus, the no-delivery duration may coincide with no current output, rather than reverse current.

FIG. 5B illustrates another embodiment in which the alternating power signal outputs a symmetrical square wave. FIG. 5B (and other waveforms illustrated herein) illustrate use of charged balance alternating currents. One example of such charged balanced alternating currents includes symmetrical waveforms in polarity. Depending on the application, the cycle may be long (e.g., 20 minutes) or short (1/60 of a second). The delivery duration may correspond to half of the period of the waveform. In the implementation shown, a reverse current is used to in the non-delivery duration, to actively prevent agent delivery to the skin layer.

FIG. 5C illustrates another embodiment in which the alternating power signal outputs an asymmetrical square wave, in that the delivery duration is different than the non-delivery duration. More specifically, the asymmetrical square wave may include longer delivery durations (t₁), followed by short(er) rest durations (t₂). The rest durations may correspond to periods of no current, or as shown, reverse current (I₂). In one application, the rest duration enables the skin layer to recuperate from the drug delivery in the prior duration (e.g., to dissipate any heat, concentration of ions, or other by products resulting from the delivery of current). As an alternative or variation, the rest period may follow a period where no current is applied to the skin layer, so as to enable the skin layer to recuperate from application of current.

FIG. 5D illustrates another embodiment in which the alternating power signal is trapezoidal, so as to include a ramp-up and/or ramp-down. As depicted, I₁ is the maximum current output generated from the power source 308. The ramp-up period extends for a duration tᵣ, selected for reasons that include enabling the user to physically accustom to the application of current and/or active agent. The period may be long, to enable the ramp-up duration to be effective. In an embodiment, a ramp-down period may optionally be implemented.

FIG. 5E and FIG. 5F illustrate alternative waveform variations in which high-frequency oscillations are superimposed on a base waveform. The base waveform may have a period that lasts seconds or minutes, corresponding to output current to the electrode assemblies ranging from a maximum (e.g., 4 MA) to no current and/or reverse current. The high-frequency oscillations reflect small variations in the current value at instances in the period. The period of the high-frequency oscillations may be one or more magnitudes shorter than that of the base waveform. As an example, the base waveform may have a period ranging seconds to minutes, and the high-frequency oscillations of the waveform may have a period that ranges between milliseconds and seconds. The effect of the high-frequency oscillations is to reduce the effects of the capacitive charge in the skin layer when receiving the active agent. The high frequency oscillations may also be used to facilitate transport of the active agent through the skin including through the stratum corneum layer (the upper most layer of the epidermis, also known as the horny layer) by causing oscillations in the movement of the active agent as it travels through the skin so as to find pathways of least resistance through the skin. In such embodiments, the high frequency oscillations may be adjusted to enhance this effect through use of modeling (e.g., pharmacokinetic modeling) and/or the patient's age, skin type and skin location

The base waveform may be selected for considerations such as described in prior embodiments. For example, in FIG. 5E, the waveform includes a ramp-up time period. In FIG. 5F, the waveform has a delivery duration that is switched to a non-delivery duration. The embodiment of FIG. 5F illustrates that the high-frequency oscillations may be generated to be present only during the delivery duration.

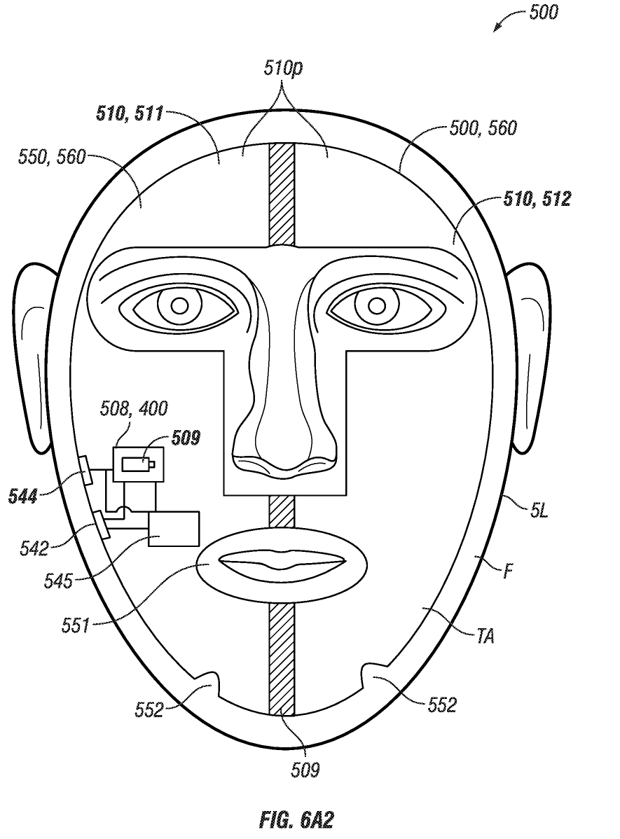

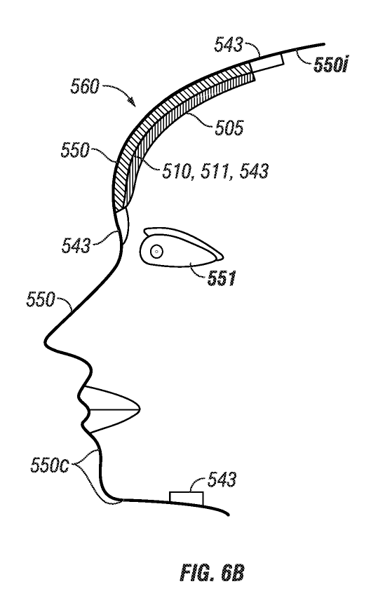

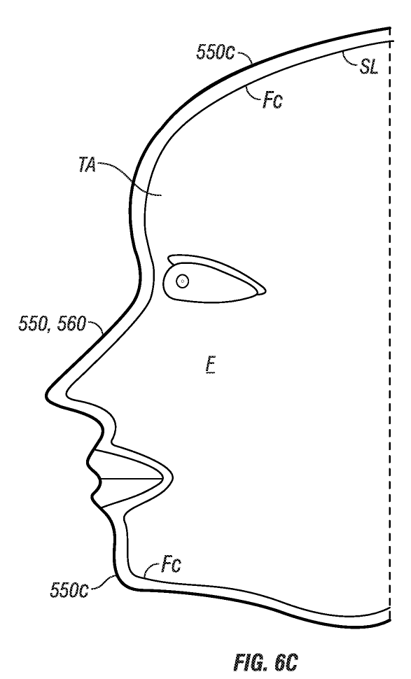

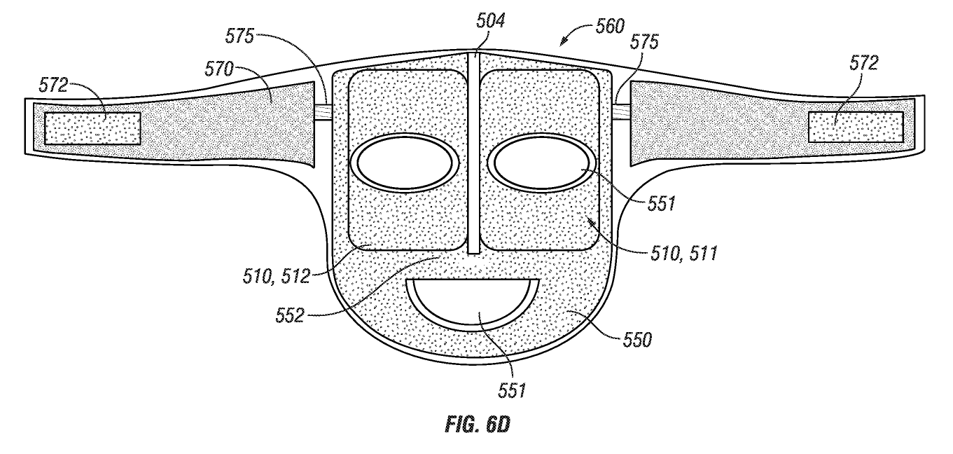

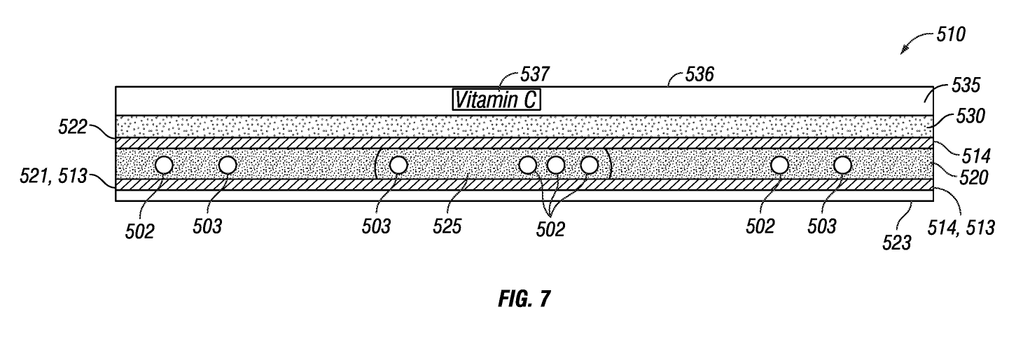

Referring now to FIGS. 6-10, in many embodiments, the invention provides a system 500 for the transdermal delivery of active agents 202 including cosmetic agents 502 (shown in FIG. 7) for the treatment of the patient's skin on one or more locations on the patient's body including, for example, the face, head and neck. System 500 includes a power source 508 such as power source 108 described above and at least one electrode assembly 510. The power source provides an output current that alternates between a maximum current value and a minimum current value. In many embodiments, the system includes a first and second electrode assembly 511 and 512 comprising a pair 510p of electrode assemblies 510. Each electrode assembly 510 of the pair 510p is configured to be held in contact with a skin layer SL of a user such as that on the user's face F. Additionally, each electrode assembly 510 can include an electrode 530 (shown in FIG. 7) that is operably coupled to the power source 508 to receive the output current from the power source. In some embodiments the power source is external to mask 550 as shown in FIG. 6A1 and connectable to the electrode assemblies via insulated wire or other electrical coupling means. According to other embodiments, the power source 508 is integral or otherwise directly coupled to the mask as shown in FIG. 6A2 and is directly or operably coupled to electrode assemblies 510 (e.g., by means of microprocessor or other electronic controller 545, which also correspond to interface 545). In such embodiments, the power source 508 may include a battery 509, such a lithium ion battery as well as one or more of a waveform generator 400 which may include a power inverter 410 and waveform shaper 420 as described above with respect to the embodiment of FIG. 4. The power source 509 may also be coupled to a user input mechanism 542 (e.g., a button) similar to input mechanism 342, which is disposed on the top surface 535 (shown in FIG. 7) of the facemask assembly 560 to allow the user to start and/or stop the flow current to electrode assemblies 510 at the user's election. For example, the user may position the facemask assembly on his or her facial, then press a button 542 to cause the power source to power the electrode assemblies to start the delivery of cosmetic agent 502 and the press the same or a different button to depower the electrodes and stop the delivery of cosmetic agent. Desirably, the button or other input mechanism 542 is configured and positioned on the facemask assembly 560 so that the user can actuate the button when the mask is on their face and can easily feel where it is (e.g., through the use of a texture on the button or hepatic sensor and feedback) or easily see in it when looking in a mirror.

The power source 508 may also be coupled to one or more sensors 544, corresponding to sensors 344 described above for detecting when the facemask assembly has been applied and made contact (e.g., electrical or physical) with the user's face. Similar to the embodiment of FIG. 3, the output of sensor 544 may then be used to initiate current delivery to the electrodes by the power source 508 and the subsequent delivery of cosmetic agent 502 (shown in FIG. 7). In various embodiments, one or more of the power source 508, input mechanism 542, and sensors 544 may also be coupled to an interface 545 which may correspond to the interface 345 of the embodiment of FIG. 3 described above. In various embodiments, interface 545 may include or correspond a microprocessor or other electronic controller either analogue or digital. It may also include a display. The interface 545 may also include logic for analyzing input from sensors 544, e.g., to determine when the face mask assembly has been applied or removed from the user's face and then start or stop the delivery of current to electrode assemblies 510 based on that determination. It may also include logic for making various skin impedance measurements and output a signal encoding an instruction to the user to perform a moisturization or exfoliation pretreatment of their skin as described herein. The output signal may be sent to a display on the interface or to a remote device such as a cell phone. Also, the interface 545 may include memory resources for storing a current regimen for a particular facemask assembly/cosmetic agent and logic for implementing that current regimen via signals sent to power source 508 encoding the parameters of the current regimen (e.g. the current, voltage frequency, waveform and charge balance of the waveform).

At least one of the electrode assemblies 510 in the pair 510p includes a medium 540 such as a hydrogel for example, that carries or otherwise comprises a cosmetic agent 502 (shown in FIG. 7) or other active agent having a charge. The medium is provided in or on the at least one electrode assembly to enable the output current of the power source 508 to repel or otherwise propel the cosmetic agent into the skin layer for a duration in which the output current has a polarity that is the same as a polarity of the active agent. According to one or more embodiments, the output current of power source 508 is a charged balanced alternating current (AC) output as described above.

The structure of an embodiment of electrode assembly 510 for delivering a cosmetic agent 502 to the user's skin will now be described in FIG. 7. It should be appreciated that this structure is exemplary and other structures and arrangements are also contemplated. The electrode assemblies 510 will typically comprise a matrix layer 520 which contacts tissue, an electrode 530 and an insulative layer 535 above the electrode. According to one or more embodiments, matrix layer 520 comprises a hydrogel material which may be pre-loaded with cosmetic agent 502 at the factory or may be post loaded by the user. The electrode 530 will typically comprise graphite or other conductive material that is sandwiched between the hydrogel and the insulative layer. Desirably, the electrode 530 is sufficiently flexible to allow the facemask assembly 560 to bend and flex with movement of the user's face to allow electrode assemblies 511 and 512 to maintain full electrical contact with the user's skin including during periods of current delivery. In use, such embodiments prevent or reduce the likelihood of any reduction in the amount of cosmetic agent delivered due to the loss of such contact. Such flexibility can be obtained by the use of a thin conductive metal material, such as a flexible graphite material, or a conductive polymer known in the art for electrode 530. The insulative layer 535 is located above the electrode 530 and may comprise any insulative material known in the polymer and/or medical electronics arts. It may also include indicia 537 or other markings on its top surface 536 (e.g., facing away from tissue) indicating what the cosmetic agents(s) is/are in a particular facemask assembly 560, as well as the dosage of the cosmetic agent.

Matrix layer 520 may comprise any material that is capable of being hydrated or otherwise storing water in bound or unbound form, as well as storing cosmetic agent 502 within the matrix. For ease of discussion, matrix layer 520 will now be described as hydrogel layer 520, but other matrix like materials are contemplated such as various polymer gels known in the biomaterials and polymer arts. Hydrogel layer 520 includes a tissue contacting side 521 and an opposing side 522 which is operatively coupled to electrode 530 either directly (typically) or indirectly. Typically, layer 520 will be configured to be sticky on both sides so as to stick to both the skin as well as the electrode. This can be implemented through the use of various adhesives known in the medical and adhesive arts which may be applied as a coating or layer 514 onto sides 521 and 522 of hydrogel layer 520. In these and related embodiments, tissue contacting side 521 may include a protective pealable (or otherwise removable) layer 523 which the user removes prior to use. Desirably, hydrogel layer 520 is impregnated or otherwise contains the cosmetic agent 502 or other active agent 102. As discussed above, the cosmetic agent 502 can be preloaded at the factory or later by the user using an application means such as a bottle of cosmetic agent containing a dispensing tip or other applicator. Layer 520 will also typically contain a liquid carrier medium 525 such as an aqueous solution in which the cosmetic agent 502 is or becomes dissolved. In these and related embodiments, the hydrogel layer 520 may include a pH buffering agent 503 to maintain a neutral pH of the carrier medium. In some embodiments, the electrode assembly 510 including the hydrogel or other matrix layer 520 may be fluidically coupled to a separate or external reservoir (not shown) containing the medium, buffering agents and one or more electrolytes to increase the conductivity of the solution. As an additional feature in such embodiments, the assembly 510 can include a releasable tab or seal which the user pulls before use to allow fluid to flow from the separate reservoir into the hydrogel layer 520. Once there, the hydrogel wets and swells with the solution. In one implementation, the cosmetic agent 502 is still contained in the hydrogel layer 520 and then dissolves in the carrier medium once the medium reaches the hydrogel. In another implementation, the cosmetic agent 502 along with the buffering agents 503 are pre-dissolved in the carrier medium 525 in the separate reservoir and the mixed solution flows into and wets the hydrogel within layer 520.

In many embodiments, electrode assemblies 510, such as assemblies 511 and 512 may be positioned in or on a mask 550 (herein facemask 550) shaped and configured to fit over all or a portion of a user's face. The facemask will typically include openings 551 for one or more of the user's eyes and mouth. It may also include notches 552 or size accommodation feature positioned on the mask for accommodating different faces sizes and the shape and protrusion of the user's nose. Typically, the electrode assemblies will be positioned on an inner surface 550i of the mask 550 (shown in FIG. 6B) and may be incorporated and/or integral to the structure. Collectively, the facemask 550 and the electrodes assemblies 510, such as assemblies 511 and 512 will now sometimes be referred to as a facemask assembly 560. The area of the user's face contacted by one or more electrode assemblies 510 on mask assembly 560 will sometimes be referred to herein as treatment area TA. Desirably, the face mask assembly 560 is configured to conform to the contour of at least a portion of the user's face F including that over treatment area TA. Such conformability can be accomplished by constructing facemask 550 from conformable polymers known in the art, such as various elastomers including, for example, polyurethane, silicone and copolymers and foams thereof. Other conformable materials area also considered such as conformable textiles. Portions of electrode assemblies 510 can be constructed from similar materials as well. In some embodiments, mask assembly 560 can be configured to be held onto the user's face by the use of an adhesive coating or layer 514 placed on the skin contacting surface 513 of electrode assemblies 510 as is explained below (similar coatings may also be placed on all or a portion of mask inner surface 550i). In such embodiments, after the user places the mask assembly 560 on his or her face, they then press on the mask to secure the mask in place to their face. In alternative or additional embodiments, mask assembly 560 may include a strap 570 to hold the assembly 560 in place. Strap 570 may be self-adjusting (e.g., by use of self-adjusting elements 572), so as to allow the user to adjust how tightly the mask fits over the user's face including the amount of pressure/force applied by assembly 560 to the skin of the user's face. Adjustment elements 572 may correspond to one or more of VELCRO elements or other fastening elements as shown in FIG. 6D. In particular embodiments, the strap 570 may include force/pressure measurement means 575 such as various strain gauges known in the art to allow the user to know and adjust the specific amount of force/pressure applied by the mask assembly 560 to their face. In use, such embodiments can serve to make sure that the mask assembly 560 makes uniform contact with the user's skin over the tissue contacting surface 561 of the mask assembly 560 including portions of the mask containing electrode assemblies 510. This, in turn, provides for a more uniform delivery of cosmetic agent 502 to the treatment area TA of the user's face and in turn a more uniform cosmetic effect from the particular cosmetic agent 502. It also reduces areas, referred to as dead areas, where electrode assemblies 510 do not make electrical contact, or only make partial contact with the user's skin which may result in increased current densities in the contacted area's skin in the treatment area TA possibly resulting in skin irritation and/or thermal, electrical or other injury to the skin from excessive current delivery. Thus, in use, embodiments of invention which provide a strap 570 and a force/pressure measurement means 575 also provide the added benefit of reduced or eliminated risk of injury to the skin from iontophoretic delivery of cosmetic agent 502 or other therapeutic agent 102 to the face F or other areas of the user's skin. Similar results may be obtained by the selection use of adhesive coatings or layer 514 for electrode assemblies 510 (or areas around the electrodes) which is arranged and configured to uniformly secure mask assembly 560 to the user's face F.

In some embodiments, such as the embodiment of FIG. 6C, the shape or contour 550c of mask 550 can be a generic shape configured to fit most human faces, particularly in embodiments where the mask is fabricated from conformable materials allowing the user to press fit the mask against their face so as to assume the contour Fc of that individual's face. According to other embodiments, mask 550 can come in a variety of sizes and shapes which the user can then select from based on their individual facial features. For example, there may be small, medium and large sizes, corresponding, for example, to the average facial size for the medium size, and one standard deviation below average size for the small and one standard deviation above average for the large.

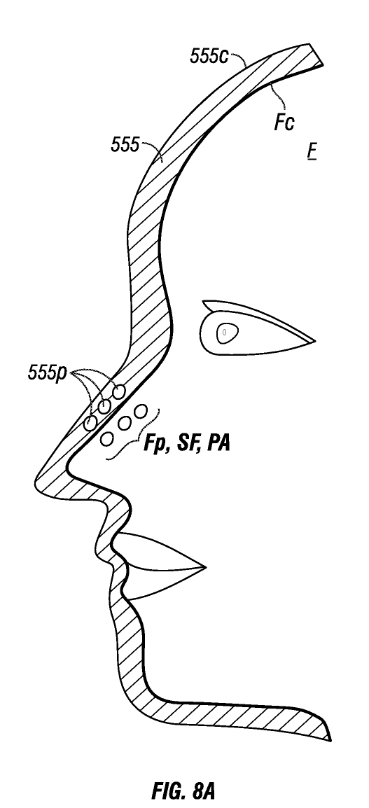

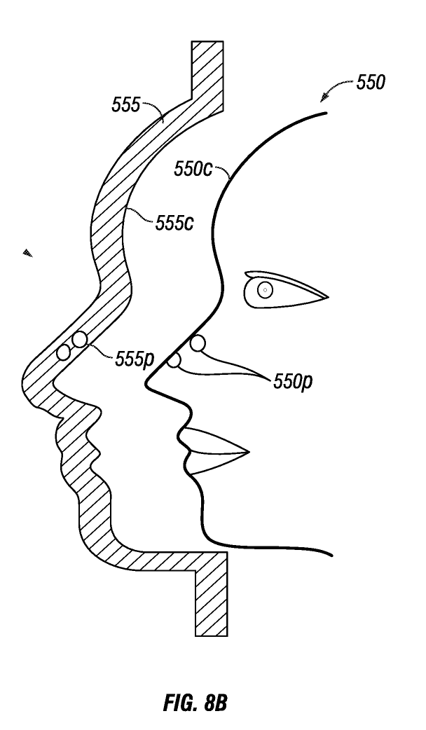

In still other embodiments, mask 550 and mask assembly 560 can be custom fit to the user's face. The custom fit can include one or more of the size, contour or included features such as a pore structure feature described herein which align with and are used to treat features on the users face. Such customization can be accomplished by a variety of means. For example, according to one embodiment, illustrated in FIGS. 8A-8B, a mold 555 can be made of the user's face F using compliant and/or conformable mold making materials known in the art. Such materials being pressed against the user's face and then lifted off to form mold 555. Suitable conformable materials including various paraffins and/or elastomers (e.g., silicone) or conformable textiles impregnated with a curable paste, polymer or like material (e.g., Plaster of Paris mold materials, etc.). The mold can be used to make the mask 555 by standard molding methods known in the polymer and molding arts.

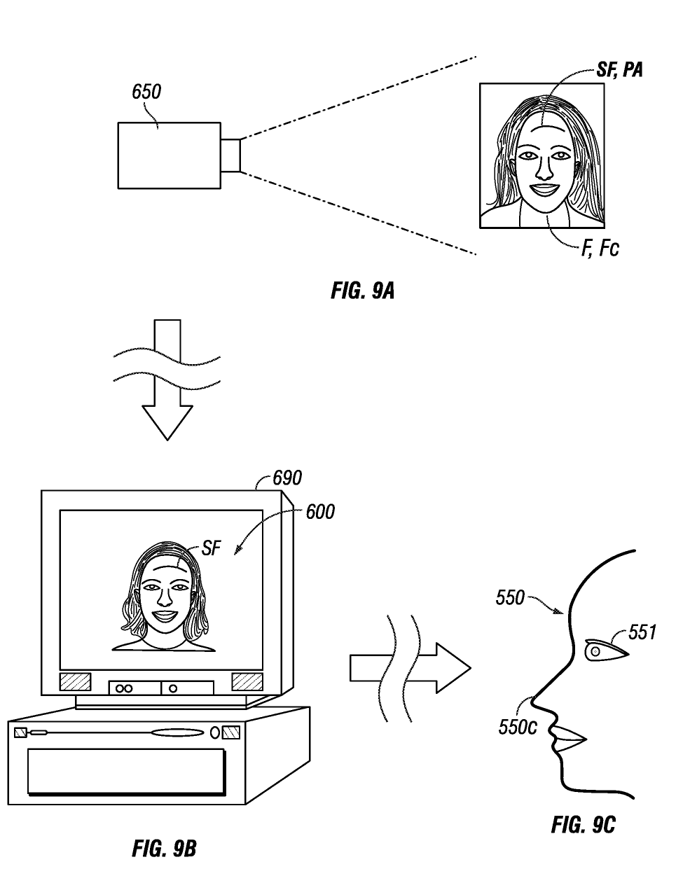

According to another embodiment, illustrated in FIGS. 9A-9C, one or more digital or other photographic images 600 can be taken of the user's face F using a digital camera, cell phone or other imaging device 650 and then that image can then be transferred to a computer or other image processing means 690 and used to construct mask 550 using fabrication methods known in the polymer and custom fabrication arts, such as 3-D printing. In particular embodiments, the mold 555 and/or digital image 600 can be used to capture not only information on the shape of the user's face, but also that of one or more skin features SF including, for example, pore structure (e.g. large pores), hair follicles, wrinkles, and pigmentation and/or hyperpigmentation areas (e.g., age spots) and other particular areas PA in the user's face to be treated. In particular embodiments, the mold 555 or image 600 may be configured to capture the pore structure Fp of different areas of the patient's face. In the case of the mold, this can be done by the intrusion of the mold material into pore structure Fp so as to have a pore structure or other mask feature 555p in the mold which can be used to produce a pore structure feature 550p in the mask assembly 560 (including in electrode assembly 510) which desirably aligns with a particular area PA on the user's face having a particular pore structure Fp when the mask is placed on the user's face. As is explained below, pore structure feature 555p can be used to selectively treat particular area PA including skin features SF in that area. The topography or contour 555c of the mold 555 including that of pore structure feature 555p may then be digitized using digital or other imaging modality such as imaging device 650. The information derived from the digital image and/or mold on the contour FC of the user's face and skin features SF in particular areas PA can then be used to adjust and/or more precisely control the delivery of cosmetic agent 502 to those particular areas PA having skin features SF to be treated so as to obtain an improved cosmetic result in those areas. Such an approach can be achieved by one or more means including, for example, the following: i) customization of the current regimen (e.g., waveform, current, voltage charge balance, etc.) delivered to areas PA and/or; ii) control of the type and amount of cosmetic agent 502 preloaded into the portions 513 of electrode assemblies 510 that are configured to make contact with areas PA. In specific embodiments of means i), the patient's pore structure can be used to make a determination of the permeability of the skin to a particular cosmetic agent which in turn can be used to adjust one or more parameters of the current regimen including, for example, the amplitude frequency and waveform shape of the current. For example, increased current and/or voltage may be used for a patient's skin having smaller and/or fewer pores and vice versa. According to one embodiment, the current for smaller pores and/or areas of reduced pore density may be in the range of about 0.4 to 0.6 mA, while for larger pores and/or higher pore density (e.g., more numerous pores) the current can be in the 0.2 to 0.4 mA range.

In specific embodiments of means ii), the amount of a particular cosmetic agent 502 carried or otherwise present within or on the electrode assembly may be titrated (e.g., adjusted) to treat the particular skin feature SF. This may be done either at the production facility by preloading or by the user using applicator 590. For example, for wrinkles, increased amounts of a collagen stimulating agent 502 may positioned in the precise location on mask assembly 560 corresponding to the location of the SF in the area PA. Likewise, for areas of hyper-pigmentation, increased amounts of a skin lightening agent 502 may be similarly positioned on the mask assembly 560. In various embodiments, the increased amounts of the particular cosmetic agent may correspond to one or more of 10, 20, 25, 30, 40, 50, 75, 100, 200, 250 or 300 percent more from that typically used (e.g., that amount used when applied externally on the surface of the skin by hand using commercially available cosmetic agents) with even larger amounts contemplated. In use, such an approach provides several benefits including: i) the ability to titrate the amount of cosmetic agent delivered to those particular areas PA to treat the particular skin features SF; and ii) the ability to titrate the amount of cosmetic agent 502 delivered so as to account for variations in the skin permeability in areas PA to agent 502 due to variations in pore structure. Both of the preceding factors in turn can provide for improved cosmetic outcomes for the user.

With regard to the use of pore structure feature 550p to treat particular skin are PA, in various embodiments, this may be accomplished by configuring the pore structure feature 550p when it is part of electrode assembly 510 to have a different conductivity and/or be loaded with different amounts of cosmetic agent 501 so as titrate the delivery of cosmetic agent to particular area Fp including to treat a selected skin feature SF in that area (e.g., wrinkle, pigmented area, inflamed area, dry area, hair follicle, etc.).

In various embodiments, one or more electrode assemblies 510 can be positioned on mask 550 (or another supporting structure) to fit over and make contact with selected areas of the user's face F such as the nose, forehead, cheeks, chin etc. In particular embodiments, electrode assemblies 511 and 512 can be positioned on mask 550 (or other supporting structure) to fit over and make contact with the left FL and right FR half of the user's face F. In these and related embodiments, assemblies 511 and 512 may be separated by an insulative barrier 509 which may be fabricated from an insulative material such as silicone or other insulative polymer known in the art. Typically, the insulative barrier 509 will have a vertical orientation as shown in the embodiment of FIG. 6 but other orientations are also contemplated. In additional or alternative embodiments, electrode assemblies 511 and 512 are arranged vertically on mask 550 so as to cover upper and lower regions of the user's face such as the forehead and below the forehead. In such embodiments, insulative barrier 509 will have a horizontal orientation so as to separate upper and lower arranged electrode assemblies 511 and 512.

A discussion will now be presented of various cosmetic agents 501 which may be delivered by embodiments of the invention including those having electrode assemblies 510 and mask assemblies 560. It should be appreciated that this list is not exhaustive and other cosmetic agents not mentioned are also contemplated. Also embodiments specifically contemplate the combination of one or more of the listed cosmetic agents. In various embodiments, the cosmetic agent 502 delivered by embodiments of the invention may correspond to one or more of various moisturizing agents, anti-oxidants, anti-wrinkle agents, collagen stimulating agents, skin lightening agents, exfoliating agents, acne treatment agents, skin ablating agents, sun block agents, depilatory agents and combinations thereof. It may also correspond to agents having a plurality of the aforementioned affects. Example anti-oxidants include vitamin A (retinol) vitamin C (ascorbic acid), vitamin B3 (Niacinamide) vitamin E (α-tocopherol), alfa-lipoic acid, carnosine (including L carnosine), resveratrol, green tea, lutein and retinol. In various embodiments, combinations of anti-oxidants or other cosmetic agents may be delivered by one or more electrode assembly 510 so as to produce an enhanced or synergistic anti-oxidant or other cosmetic effect including a synergistic skin rejuvenating effect such as skin lightening, increased skin elasticity, etc. An example of such a synergistic antioxidant combination includes vitamin C and vitamin E and the effects include anti-oxidation, skin lightening and UV protection. Another example of a synergistic combination of cosmetic agents includes alfa lipoic acid and L-carnosine, where the effect is both anti-oxidation and also anti-inflammatory including reduction in skin redness (erythema) due to rashes and other causes. Example collagen stimulating agents include, for example, vitamin C and various polypeptides including carnosine, N-Acetylcarnosine, Trifluoroacetyl-Tripeptide-2, Tripeptide-10 Citrulline and various Palmitoyl Tripeptides (e.g, 1, 3/5 and 38). Example hydrating agents include hyaluronic acid, vitamin C and hydrolyzed glycosaminoglycans. Example skin ablating agents include α-β-lipo-hydroxy acids. Example sun block agents include zinc oxide. Example depilatory agents include eflornithine.

In other particular embodiments, the cosmetic agent 502 may comprise one or more peptides having neuromuscular inhibitor effects on the motor and other neurons which innervate the facial region so as to reduce the appearance of lines and wrinkles by relaxing muscles in the face which causes the wrinkles. One example of such a motor neuron inhibiting agent includes Botulinum toxin, an example of which includes BOTOX, available from the Allergan Corporation. Other examples include neuromuscular inhibiting peptides found in various snake venoms.

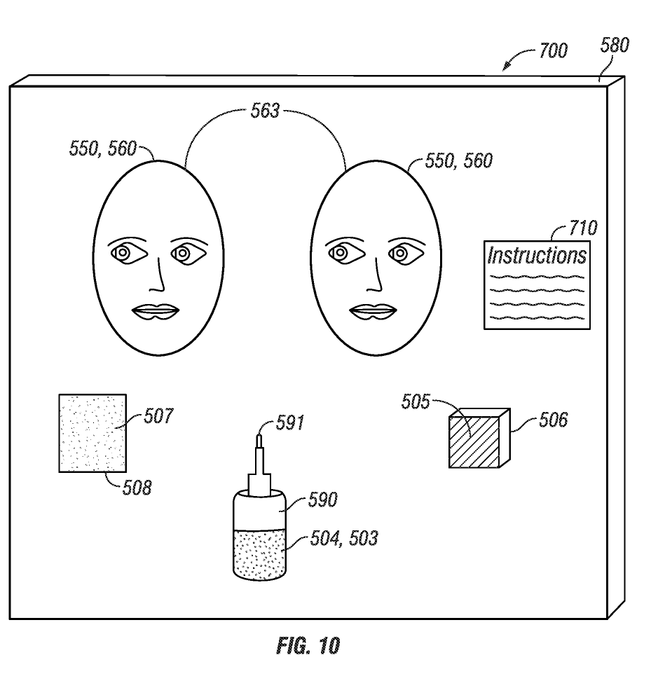

In preferred embodiments, one or more of the above or other cosmetic agents 502 may be preloaded into the electrode assembly 510/facemask assembly 560. In these and related embodiments, the user then selects the facemask assembly 560 containing the desired cosmetic agent or agents 502. Referring now to FIG. 10, in alternative or additional embodiments, the user may load the cosmetic agent 502 or a cosmetic agent solution 504 into the electrode and/or facemask assembly 510/560. In such embodiments, the cosmetic agent 502 cosmetic agent solution 504 can be contained in a bottle or other container 590 which may have a tapered dispensing tip or other dispensing element 591 configured to be fluidically coupled to one or more of the electrode assembly 510, hydrogel layer 520 or a cosmetic agent or agent solution reservoir.

In addition to facemask assembly 560, various embodiments of the invention also provide a kit 700 comprising an embodiment of the facemask assembly 560 which is preloaded with one or more cosmetic agents 502 described herein and is packaged in sealed packaging 580. In some embodiments the kit 700 may include instructions for use 710 of system 500 including for facemask assembly 560. Also in some embodiments, the kit 700 may a bottle or other container 590 of cosmetic agent 502 (or cosmetic agent solution 504), such as that described above, which is used to load cosmetic agent 502 or cosmetic agent solution 504 into the facemask or electrode assembly. Typically in these embodiments, electrode assemblies 510 (e.g., assemblies 511 and 512) are not preloaded with cosmetic agent 502, but in some case they maybe and bottle 590 may be used to reload the electrode assemblies with cosmetic agent after a delivery regimen/period of cosmetic agent has been performed so as to allow the facemask assembly to be reused. The kit 700 may include a single facemask assembly 560 containing one or more cosmetic agents 502 or a plurality of facemask assemblies 563 which comprise the same or different cosmetic agents 502 or the same cosmetic agent 502 in varying amounts. For embodiments providing a plurality of face mask assemblies 563, the facemask assemblies 560 in the plurality may be configured to provide a facial treatment regimen. In such embodiments, the treatment may comprise the serial application of facemasks and delivery of selected cosmetic agents 502 over a selected period of time. The facial treatment regimen may be implemented through the selection of the particular cosmetic agent and/or the dose of cosmetic agent.

Other embodiments of the invention provide methods for iontophoretically delivering a cosmetic agent 502 or other active agent 102 to the skin of the user's face or other location on their body. In many embodiments, this may be done using an embodiment of the facemask assembly 560 described. According to one such embodiment for doing so, the user chooses a facemask assembly 560 which may be preloaded with selected cosmetic agents 502 or they may load the assembly including electrode assemblies 510 with the desired cosmetic agent 502 using bottle 590 or other loading means. They then apply the assembly 560 to their face, which may involve pressing the facemask assembly firmly to the skin of their face so as to have the adhesive layer 514 of the mask assembly stick to their facial skin and/or adjusting strap 570 to achieve a desired tightness of fit which can be quantified using force measurement means 575. They may then select (e.g., using input mechanism 542), a delivery regimen stored on waveform generator 400 or interface 545 and begin iontophoretic transdermal delivery of the cosmetic. The delivery regimen may include one or more of delivery period, delivery current and waveform shape. Typically, the cosmetic agent 502 will be delivered with a pair 510p of electrode assemblies 510 such as assemblies 511 and 512 using double point dispersion approach described herein. However, in some embodiments, the cosmetic agent 502 is delivered using only one electrode assembly using a single point dispersion approach. In one or more implementations of double point dispersion, the operation of the electrode assemblies 510 is configured such that the delivery of cosmetic agent at each electrode assembly (e.g., into area of the skin contacted by the electrode assemblies) is substantially equivalent. In particular implementations, this can be accomplished by configuring the on and off periods of operation each electrode to be substantially equivalent. In other approaches it may accomplished by measuring the impedance through each electrode assembly 510 and then using waveform generator 400, to adjust the on and off periods or current characteristics (e.g., amplitude, frequency etc.) for each electrode assembly to compensate for differences in impedance at each electrode assembly that may affect the amount of cosmetic agent delivered to the skin contacted by each assembly.

In one or more embodiments, the user may apply the facemask assembly 560 and/or other structure holding electrode assemblies 510 directly to their skin without pretreatment or they may pretreat their skin prior to the application of the facemask assembly. According to one embodiment of pre-treatment, the user may treat their skin with a moisturizing agent 505 such as those described herein or otherwise known in the art prior to application of the facemask assembly 560 and subsequent to delivery of the current by the power source 508, 108. The pretreatment with the moisturizer herein, described as pre-moisturization or a pre-hydration step, serves to reduce the impedance of the skin and in turn reduces the amount of resistive heating of the skin. In use, such embodiments reduce the risk of thermal irritation and/or injury to the skin as well as any resulting erythema or other discoloration of the skin. The moisturizing agent 505 may be included with kit 700 in a packet 506 or it may be included with facemask assembly 560 as a surface layer that it is coated onto the tissue contacting side 513 of the electrode assembly 510 or the user may use their own moisturizing agent. In some embodiments, the user may measure the impedance and/or level of hydration of the target skin site to be treated and then, use that information to make a determination as to whether to apply the moisturizing agent and for how long. The skin impedance level can be measured using the electrode assemblies 510 included with the face mask assembly 560 or by a separate skin impedance/hydration measurement sensor 565 which is coupled to the mask or is positioned on a separate structure. In use, embodiments of the invention which utilize such skin impedance measurements provide the user with the ability to know when and for how long to apply a moisturizing agent so as to increase the delivery of the cosmetic agent 502 during a selected delivery period and reduce or eliminate any thermal or electrical or other injury to the skin resulting from the current used for iontophoretic delivery of the selected cosmetic agent to the skin.

In other embodiments, the user may treat the skin prior to the application of the facemask assembly 560 and current delivery by exfoliating the skin using an exfoliating agent 507 known in the art to remove the dead layer of cells in the uppermost layer of skin, e.g., the stratum corneum SC so as to increase the permeability of the skin to the cosmetic agent 502 as well reduce skin impedance, both serving to increase the transport of cosmetic agent 502 into the skin and in turn enhance the desired cosmetic effect. The exfoliating agent 507 may be supplied with the kit along with a specific cloth 508 to use for doing the exfoliation which may have the exfoliating agent 507 embedded or otherwise pre-applied to it. Similar to the pre-moisturization step, prior to or after exfoliation, the user may make measurements of skin impedance using the face mask assembly 560 or external impedance sensor to determine when a sufficient level of exfoliation has been obtained. In use, embodiments using pre exfoliation serve to increase the delivery of cosmetic agent 502 into the skin as well to do so more uniformly in the area of skin contacted by the face mask assembly 560.

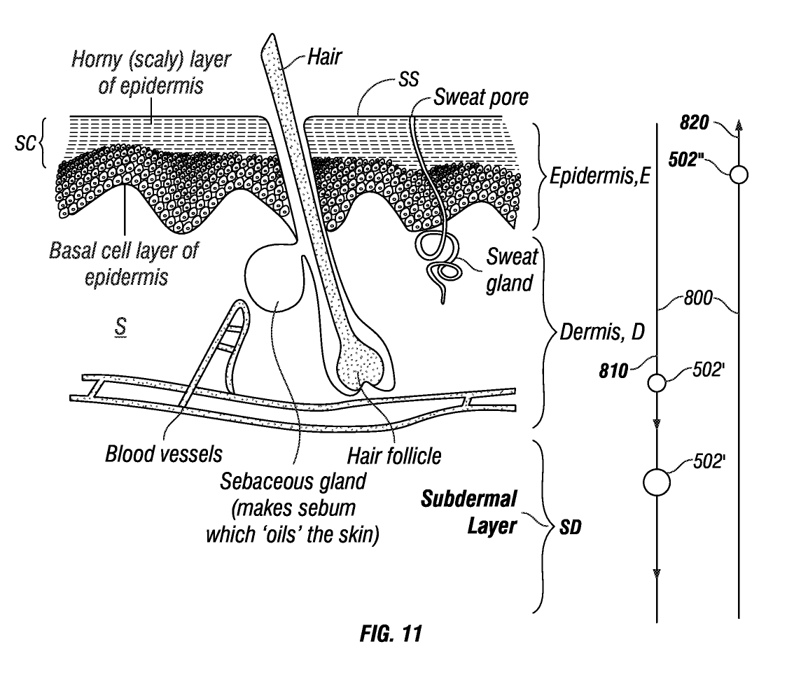

Various embodiments of the invention including those using facemask assembly 560 provide for the ability to iontophoretically deliver a cosmetic agent 502 into the skin including into selected layer of the skin such as the epidermis or dermis. Further embodiments using iontophoretic methods described herein provide for the ability to deliver sufficient amounts of a cosmetic agent through stratum corneum (SC) barrier layer of the skin (also known as the horny layer) and into a selected inferior layer of the skin (e.g., the dermis) in order to produce a desired cosmetic effect. In particular applications, embodiments of the invention can be configured to deliver effective amounts of cosmetic agent to a selected depth into the skin such as the depths corresponding to the epidermis E (e.g., 0.08 to 0.012 inches) or the dermis D (e.g., 0.012 to 0.016 inches) or other skin layer as shown in FIG. 11. Such delivery methods provide several advantages. First, one or more cosmetic agents 502 are delivered into a layer of the skin such as the epidermis or dermis where it can have the most significant cosmetic effect (e.g., anti-oxidant, collagen stimulating, moisturizing effects, etc.). Second, as the cosmetic agent is delivered beneath the surface of the skin (in particular beneath the stratum corneum barrier layer of the skin), appreciable amounts of the cosmetic can be delivered to those inferior layers to have the desired cosmetic effect. Third, once delivered to those lower layers, owing to the barrier function of the upper stratum corneum layer, the cosmetic agent can remain there for an extended period of time (e.g., hours to days) to have a longer acting and/or prolonged cosmetic affect (e.g., hours to days) as opposed to only being applied to the surface of the skin where it may be washed away or otherwise wear off or never even penetrate in significant quantities. In further approaches for iontophoretically delivering cosmetic agents into the facial or other skin area of the user, embodiments of the invention can also be configured to deliver cosmetic agent into the user's skin so as produce a vertical concentration gradient 800 of cosmetic agent 502 in the user's skin S including epidermal, dermal and even subdermal layers E, D and SD. In some embodiments, the gradient can be an increasing vertical gradient 810 with the higher concentration at the deepest levels of the skin (e.g., dermal or subdermal layers). In use, such embodiments provide the greatest cosmetic effect at the deepest levels of the skin, e.g., the dermal or subdermal layer. In other embodiments, the gradient can be a decreasing gradient 820 with the highest concentrations at the uppermost portions of the skin, such as the epidermis E. These embodiments provide the greatest cosmetic effects closest to the skin surface SS. In additional or alternative approaches, embodiments of the invention can be used to produce an increasing gradient 810 for one particular cosmetic agent 502′ (e.g., a collagen stimulating agent) and a decreasing gradient 820 for another particular cosmetic agent 502″ (e.g., an antioxidant). Such embodiments can be used to produce desired cosmetic effects in both the upper and lower portions of the skin. Increasing gradients of cosmetic agent can be achieved by applying current to the electrode assemblies (e.g., those in those in the facemask assembly 560) for longer periods of time (e.g., 20, 30, 60 minutes or even longer) to drive the cosmetic agent deeper into the skin, while the reverse is true for decreasing gradients. In alternative or additional embodiments, a combination of concentration gradients can achieved by applying a first mask assembly having a first cosmetic agent and delivering current to its electrode assemblies for an extended period of time sufficient to produce an increasing gradient and then applying a second face mask assembly having a second cosmetic agent and applying current for a shorter period of time (e.g. less than 20 minutes, 10 minutes or even 5 minutes) so as to produce the decreasing gradient. Also, the application of the current during the second period will tend to drive the first cosmetic agent deeper producing an even more enhanced increasing concentration gradient for the first cosmetic agent. In use, such combinations of increasing and decreasing gradients can be used to achieve the same or different cosmetic effects in different layers of the skin (e.g., skin lightening in the epidermal layer and an increase in collagen content in the dermal layer) which in turn can achieve an overall enhanced skin rejuvenating effect for the user.

Applications

Numerous applications exist for embodiments of the described herein including, for example, various cosmetic applications. Table 2, lists various skin conditions and/or desired skin treatment and the cosmetic agents which may be used for such treatment using a system of electrode assemblies incorporated into facemask such as described above. Table 2 provides a similar list for various drugs and other active agents for treatment of other conditions. All the compound listed are considered charged or to have some have some residual charge. However, for those agents which do not have a net charge they can be formulated using known methods in the chemical arts, for example, by adding a charged functional group, such as a hydroxyl ion (OH⁻), a charged methyl group (CH₃⁻), or a chloride ion (Cl⁻, or a sodium ion (Na⁺). The tables further identify whether the treatment can be patient activated, sensor activated, timed, or continuous. If patient activated, a user input mechanism 342 (FIG. 3) may be operated by the user when the electrode assemblies are in the deployed state (e.g., when face mask assembly is put on the user face) to initiate operation of the electrode assemblies and delivery of the active agent. Examples of user activated applications include delivery of various cosmetic agents such as sun-screens and anti-oxidants and pain management drugs such as lidocaine or fentanyl. Sensor activated uses may incorporate use of one or more sensors 344 that interface with the user's body to determine whether a condition of the user requires treatment with the identified active agent. An example of a sensor activated application can include treatment of diabetes where the sensor is a blood glucose sensor (or other sensor means for detecting hyperglycemia) and administers a dose of insulin. Other examples of sensor activated applications may include, for example, the detection of a skin condition and the administration of cosmetic agent for treatment of the skin condition. In a specific embodiment of such a sensor activated application, the system may include a sensor for the detection of sun burn or other sun damage to the skin where the device subsequently administers a sun screen agent to the patient using an electrode assembly. The sensor for such an application may include a colorimetric or other optical sensor for detecting redness or erythema of the skin. A treatment is timed if it incorporates the timer 346 to determine when to start/stop the delivery durations.

TABLE 1

| | | Patient | Sensor | | Con- |
|---|---|---|---|---|---|
| Active Agent | Condition | Activated | Activated | Timed | tinuous |
| Insulin | Diabetes | X | X | X | |
| GLP-1/ | Diabetes | X | X | X | |
| Integrin | | | | | |
| Fe²⁺ | Anemia | | | | X |
| Sodium (Na), | Electrolyte | | | | X |
| Potassium (K) | renewal | | | | |
| Furosemide | Epilepsy | | X | X | |
| Bumetanide | Migraine | X | X | X | |
| Aspirin | Inflammation | X | X | X | |
| Ketoprophin | Arthritis | X | | | |
| Lidocaine | Pain | X | | | |
| Fentanyl | Pain | X | | | |
| Alprazolin | Anxiety/Pain | X | X | | |
| Antibiotics | Wound | | | | X |
| | Healing | | | | |

TABLE 2

| | | Patient | Sensor | | Con- |
|---|---|---|---|---|---|
| Active Agent | Condition | Activated | Activated | Timed | tinuous |
| Moisturizer | Dry skin | x | x | x | |
| Sun Block | Sun Burn | x | x | x | |
| Anti-oxidant | Skin damage | x | x | x | |
| Collagen | Skin damage, | | | x | |
| stimulating | wrinkled skin | | | | |
| agent | | | | | |
| Skin | Pigmented | x | | x | |
| Lightening | skin | | | | |
| agent | | | | | |
| Depilatory | Unwanted | x | | x | |
| agent | hair | | | | |
| Anti | Skin irritation, | | | | |
| Inflammatory | erythema and | | | | |
| agent | inflammation | | | | |
| antibiotic | acne | x | | x | |

In specific embodiments, the active agent can comprise a sufficient amount of elemental iron for the treatment of iron deficiency anemia. The amount of elemental iron can be sufficient to provide between 1 to 100 mg of elemental iron to the patient for a period of days or even weeks. In various embodiments the elemental iron can comprise ionic iron in the form of ferrous (Fe²⁺) or ferric (Fe³⁺) iron. The ionic iron can comprise an iron salt, a ferrous salt, a ferric salt, ferric pyrophosphate ferrous chloride or a combination thereof.

In other specific embodiments, the active agent can comprise one or cosmetic agents including for example, a moisturizer, an anti- oxidant, vitamin C, a collagen stimulating agent, a wrinkle reducing agent, an anti-inflammatory agent, a skin lightening agent, antibiotic and the like as discussed above. The amount of these and other cosmetic agents contained in the electrode assemblies described herein (e.g., those in the facemask assembly) can be sufficient to deliver between about 1 to 2000 mg of cosmetic agent into the skin of the user, with specific embodiments of 100, 200, 250, 500, 750, 1000, 1250, 1500, and 1750 mgs. In various embodiments in order to achieve the aforementioned delivery amounts, the amount of selected cosmetic agent in the facemask assembly can be between about 10 to 200% greater than the intended delivery amount with specific embodiments of 25, 50, 75, 100, 150 and 175%. For example, the above percentage increases may apply to an amount of an antioxidant described herein such as vitamin C or lipoic acid.

Conclusion

Although illustrative embodiments of the invention have been described in detail herein with reference to the accompanying drawings, it is to be understood that the invention is not limited to those precise embodiments. As such, many modifications and variations will be apparent to practitioners skilled in this art. Accordingly, it is intended that the scope of the invention be defined by the following claims and their equivalents. Furthermore, it is contemplated that a particular feature described either individually or as part of an embodiment can be combined with other individually described features, or parts of other embodiments, even if the other features and embodiments make no mentioned of the particular feature. This absence of describing combinations should not preclude the inventor from claiming rights to such combinations. Further still, embodiments of the invention also contemplate the exclusion or negative recitation of an element, feature, value, chemical or ingredient, wherever said element, feature, value, chemical or ingredient is positively recited.

What is claimed is:

1. A method for iontophoretic transdermal delivery of a cosmetic agent to a user's face, the method comprising:
providing a facial treatment mask, the mask configured to conform to a contour of the user's face and including at least a first and second electrode assembly, each of the electrode assemblies carrying the cosmetic agent;
applying the mask to the user's face, wherein the mask conforms to the contour of the user's face such that the first and second electrode assemblies uniformly contact the user's facial skin; and
delivering an alternating current through each of the first and second electrode assemblies to alternatively repel the cosmetic agent from the respective first and second electrode assemblies into the user's facial skin where it produces a cosmetic effect.

2. The method of claim 1, wherein mask is custom fit to the user's face, the custom fit including at least one of a mask size, contour or mask feature aligning with a skin feature on the user's face.

3. The method of claim 1, wherein the current is generated by a power source integral to the treatment mask or externally connected to it.

4. The method of claim 1, wherein the cosmetic agent is at least one of an anti-oxidant, moisturizer, collagen stimulating, wrinkle reducing, anti-skin lightening or depilating agent.

5. The method of claim 1, wherein the produced cosmetic effect is substantially uniform over at least an area of facial skin contacted by the first and second electrode assemblies.

6. The method of claim 1, wherein the cosmetic effect is produced in an area of facial skin contacted by the first electrode assembly is substantially equivalent to that produced in an area of facial skin contacted by the second electrode assembly.

7. The method of claim 1, wherein the cosmetic effect is a skin rejuvenating effect.

8. The method of claim 1, wherein the cosmetic effect is an increased level of skin hydration.

9. The method of claim 1, wherein the cosmetic effect is a reduced size or appearance of wrinkles on the user's face.

10. The method of claim 1, wherein the cosmetic effect is an increased level of skin collagen in the skin.

11. The method of claim 1, wherein the cosmetic effect is an increased thickness of the skin.

12. The method of claim 1, wherein the cosmetic effect is a lightening of at least one of an area of skin pigmentation, hyper-pigmentation or discoloration on the user's face.

13. The method of claim 1, wherein the cosmetic agent comprises at least a first and second cosmetic agent which are selected to synergistically interact in the skin to produce an enhanced cosmetic effect in the user's facial skin.

14. The method of claim 13, wherein the first and second cosmetic agents comprise vitamin C and vitamin E and the enhanced cosmetic effect is skin rejuvenation, anti-oxidation, skin lightening or UV protection.

15. The method of claim 13, wherein the first and second cosmetic agents comprise alfa lipoic acid and carnosine and the enhanced cosmetic effect is skin rejuvenation, anti-oxidation, reduced inflammation or reduced erythema.

16. The method of claim 1, wherein the cosmetic agent is uniformly delivered into the facial skin in an area of skin contacted by the first and second electrode assemblies and wherein the cosmetic effect is uniformly produced in the area of facial skin contacted by the first and second electrode assemblies.

17. The method of claim 1, wherein an amount of cosmetic agent delivered into an area of facial skin contacted by the first electrode assembly is substantially equivalent to that contacted by the second electrode assembly.

18. The method of claim 1, further comprising: measuring a skin impedance of the user's facial skin prior to delivery of the alternating current.

19. The method of claim 18, further comprising: using information from the skin impedance measurement to determine whether to perform a skin treatment of the user's facial skin prior to the delivery of the alternating current, wherein the skin treatment includes at least one of skin moisturization, hydration or exfoliation.

20. The method of claim 1, further comprising: applying a moisturizing or hydrating agent to the facial skin to moisturize or hydrate the skin prior to delivery of the alternating current.

21. The method of claim 20, further comprising: measuring skin impedance of the facial skin to determine a degree of skin hydration or moisturization wherein the measurement is made before or after applying the moisturizing or hydrating agent.

22. The method of claim 20, wherein current delivery is initiated after a selected skin impedance or change in skin impedance is obtained.

23. The method of claim 1, further comprising: exfoliating the facial skin prior to delivery of the alternating current.

24. The method of claim 23, further comprising measuring skin impedance of the facial skin to determine a degree of skin exfoliation.

25. The method of claim 23, wherein current delivery is initiated after a selected skin impedance or change in skin impedance is obtained.

26. The method of claim 1, wherein the current or an amount of cosmetic agent is adjusted based on a pore structure of the user's face or information from the pore structure.

27. The method of claim 26, wherein the current adjustment is in at least of an amplitude, frequency or waveform shape of the current.

28. The method of claim 26, wherein the pore structure information is at least one of an average pore size or a pore density.

29. The method of claim 28, wherein the current adjustment is a current amplitude and is inversely proportional to the average pore size or pore density.

30. The method of claim 28, wherein the adjustment the amount of cosmetic therapeutic agent carried by the electrode assembly is inversely proportional to the average pore size or pore density.

31. The method of claim 1, wherein the current or an amount of cosmetic agent carried by the electrode assembly is adjusted to treat a particular area or skin feature on the user's face.

32. The method of claim 26, wherein the current adjustment is in at least of the amplitude, frequency or waveform shape of the current.

33. The method of claim 31, wherein the particular area or skin feature is an area of discoloration, pigmentation, hyperpigmentation, redness or inflammation.

34. The method of claim 31, wherein the particular area or skin feature is a wrinkle, area of dry skin or hair follicle.

35. The method of claim 1, wherein the alternating assemblies is in a range of about 0.1 to 4 ma.

36. The method of claim 1, wherein the alternating current has a frequency in a range of about 1 mHz to about 10 mHz.

37. The method of claim 1, wherein the alternating current has waveform that is substantially sinusoidal, square, saw tooth or trapezoidal.

38. The method of claim 1, wherein the alternating current has a voltage that is in a range from about 1 to 100 volts.

39. The method of claim 1, wherein the alternating current is charged balance for at least a given duration of time.

40. The method of claim 1, further comprising:
delivering the cosmetic agent into the user's facial skin to create a vertical concentration gradient of cosmetic agent in the skin.

41. The method of claim 40, wherein the vertical concentration gradient is an increasing gradient and the cosmetic effect is greatest in a dermal or the subdermal layer of the user's facial skin.

42. The method of claim 40, wherein the vertical concentration gradient is a decreasing gradient and the cosmetic effect is greatest in an epidermal layer of the user's facial skin.

43. The method of claim 1, further comprising:
delivering the cosmetic agent into a selected layer of the user's facial skin to produce the cosmetic effect in that layer.

44. The method of claim 43, where the layer is one of an epidermal, dermal or subdermal layer.

45. The method of claim 43, where the layer is a subdermal layer and the cosmetic agent remains for an extended period of time to have a prolonged cosmetic effect.

46. The method of claim 1, further comprising:
delivering the cosmetic agent to a selected layer of the user's facial skin to produce the cosmetic effect at that depth.

47. The method of claim 46, wherein the depth is in a range of about 0.08 to 0.012 inches or about 0.012 to 0.016 inches.

48. The method of claim 1, further comprising:
sensing contact of the treatment mask with the user's facial skin and triggering the delivery of alternating current through electrode assemblies responsive to the sensed contact.

49. The method of claim 48, wherein contact is sensed by an impedance change between the electrode assemblies.

50. The method of claim 48, wherein contact is sensed by an amount of force applied from the treatment mask to the user's facial skin.

51. A method for iontophoretic transdermal delivery of a cosmetic agent to a user's face, the method comprising:
providing a facial treatment mask, the mask configured to conform to a contour of the user's face and including at least a first and second electrode assembly, each of the electrode assemblies carrying the cosmetic agent;
applying the mask to the user's face, wherein the mask conforms to the contour of the user's face such that the first and second electrode assemblies uniformly contact the user's facial skin;
delivering an alternating current through each of the first and second electrode assemblies alternatively repelling the cosmetic agent from the respective first and second electrode assemblies into the user's facial skin; wherein the cosmetic agent is uniformly delivered into the facial skin in an area of skin contacted by the first and second electrode assemblies; and
producing a cosmetic effect in the patient's facial skin, wherein the cosmetic effect is uniform over at least the area of facial skin contacted by the first and second electrode assemblies.
